(19) United States
(12) Patent Application Publication
(10) Pub. No.: US 2026/0001820 A1
(43) Pub. Date: Jan. 1, 2026

(54) PROCESS FOR THE CONVERSION OF CARBON DIOXIDE INTO VALUE-ADDED PRODUCTS BY MEANS OF A PROCESS OF MECHANOCHEMICAL ACTIVATION OF INDUSTRIAL PROCESSING SCRAPS

(71) Applicant: Universita' Degli Studi Di Sassari, Sassari (SS) (IT)

(72) Inventors: Maria Domenica SIMULA, Sassari (SS) (IT); Alessandro TARAS, Sassari (SS) (IT); Luca PINNA, Sassari (SS) (IT); Salvatore PIU, Sassari (SS) (IT); Stefano ENZO, Sassari (SS) (IT); Sebastiano GARRONI, Sassari (SS) (IT); Gabriele MULAS, Sassari (SS) (IT)

(73) Assignee: Universita' Degli Studi Di Sassari, Sassari (SS) (IT)

(21) Appl. No.: 18/878,674

(22) PCT Filed: Apr. 13, 2023
(86) PCT No.: PCT/IB2023/053778

§ 371 Date: Dec. 24, 2024

(30) Foreign Application Priority Data
Apr. 15, 2022 (IT) 102022000007670

(51) Int. Cl. C07C 1/12

(52) U.S. Cl. CPC C07C 1/12; C07C 2523/78

(57) ABSTRACT

The present invention relates to a method for converting carbon dioxide (CO₂) into high added value chemical products by means of a mechanochemical process under gas flow and continuous conditions. In particular, said process converts CO₂ into a mixture of high added value chemical compounds comprising solid products of mineral carbonation, mainly Mg, Fe and Ca carbonates, and/or low molecular weight hydrocarbons, mainly methane, ethylene and ethane. Said CO₂ conversion is achieved, in particular, by applying said mechanochemical process to slags from steelmaking processes and/or basalt scraps.

### TECHNICAL FIELD

The present invention relates to a method for converting carbon dioxide (CO₂) into high added value chemical products by means of a mechanochemical process under gas flow and continuous conditions. In particular, said process converts CO₂ into a mixture of high added value chemical compounds comprising solid products of mineral carbonation, mainly Mg, Fe and Ca carbonates, along with low molecular weight hydrocarbons, mainly methane, ethylene and ethane. Said CO₂ conversion is achieved, in particular, by applying said mechanochemical process to slags from steelmaking processes and/or basalt scraps.

### BACKGROUND ART AND TECHNICAL PROBLEM

Carbon dioxide (CO₂) is a greenhouse gas which is released into the atmosphere as a consequence of natural phenomena (such as, for example, volcanic eruptions, cellular respiration, etc.) and human activities (such as, for example, processes of combustion of organic materials of fossil and non-fossil origin (wood, oil, petrol, diesel fuel, gas, biomass, etc.), deforestation activities, etc.), the concentration of which (>400 ppm since 2015) has exceeded by 45% the maximum historical values of the pre-industrial age.^{[1][2]}

Among the direct consequences of this increased CO₂ concentration, the increase in mean global temperature, the desertification of increasingly large areas, and the occurrence of so called “extreme” and potentially catastrophic climatic events are some of the most important current environment-related issues on a global scale. Mitigating such effects requires timely interventions and positive actions by all industrialized countries.^{[3]} The carbon dioxide emission limits set by the Kyoto protocol in 1997, and then by the Paris agreement in 2015, in line with the targets of the more recent EU framework programmes for research and innovation (including Horizon 2020, Horizon Europe), have led the scientific community and industry to studying new energy sources having a low environmental impact and developing new technologies and processes aimed at reducing the levels of carbon dioxide released into the atmosphere. The attention is now being paid to reducing the use of fossil fuels, developing processes allowing for large-scale exploitation of renewable, or carbon-free, energy sources, and setting up processes and technologies for reducing CO₂ concentration in the atmosphere. In this regard, the acronym CCUS (Carbon Capture, Utilization and Storage) refers in the literature to various technologies currently under development, but still far from possible large-scale application, which are devoted to reducing atmospheric CO₂ concentration by capturing and storing CO₂ and transforming the captured carbon dioxide into products having a high commercial value.^{[4]}

In particular, much attention has been paid to Carbon Capture and Storage (CCS) procedures in recent years, with the goal of achieving, in the short term, a considerable reduction in carbon dioxide emissions by means of mineral carbonation processes occurring in geologically stable underground basins; however, the feasibility of such a process on a large-scale is currently limited by several factors, including, for example, the high costs to be incurred for sequestration, transportation, and the energy necessary for injecting CO₂-containing exhaust gas into the subsoil.^{[5]} On the other hand, the set of transformation processes (CCU) represents a virtuous way of controlling CO₂ concentration thanks to the possible creation of a so-called “zero carbon dioxide emission” scenario, since abundant CO₂ can be used as a raw material for the creation of a virtuous closed cycle, wherein the generated gas is transformed into valuable chemical compounds while at the same time balancing the atmospheric CO₂ levels.

In this latter respect, one aspect of exceptional interest from an application viewpoint concerns the experimental conditions necessary for activating the chemical processes for converting CO₂ into basic fuels and chemical products: literature data show that activation and transformation of carbon dioxide (CO₂) require severe experimental conditions to overcome the slow reaction kinetics and the high energy barrier due to its thermodynamic stability (Δ_{f}G⁰ CO₂=−394.36 kJ mol⁻¹). In general, the conversion of carbon dioxide into fuels and basic chemical substances can be exemplified by means of redox processes occurring in the presence of molecular hydrogen. It should be taken into account that hydrogen, in its turn, has long been the subject of research for potential energy vectors alternative to fossil fuels, and the search for novel processes for producing it is still underway.^{[6]} The modes of CO₂ activation reported in the scientific literature refer to processes occurring in the presence of catalysts and promoted thermally or, to a limited extent, photochemically or electrochemically.^{[7]} Catalytic processes play a crucial role in the chemical industry and will be the key for all future scenarios aimed at implementing sustainable cycles; catalysts are, in fact, compounds capable of providing a reaction path that is alternative to the original reaction path and characterized by a significantly lower activation energy barrier.

The study of transformations of natural silicates with high Ca, Fe and Mg content like, for example, Olivine, which is a mineral widely available on the earth's crust^{[8]} that has a high potential for interaction with carbon dioxide^{[9]}, can be considered to be related to both of the above-mentioned lines of intervention (i.e. CCS and CCU). Under particular pressure and temperature conditions, Olivine can, in fact, react with water (H₂O) to give the mineral known as serpentine (Mg₃Si₂O₅(OH)₄) plus a mixed oxide of Fe²⁺/Fe³⁺ and molecular H₂. This process, known as serpentinization, leads to molecular H₂ and to the partial oxidation of Fe²⁺ to Fe³⁺ into magnetite oxide. In the presence of carbon dioxide, the molecular hydrogen thus formed can, through the Fischer-Tropsch process, form light hydrocarbons, including methane, or bring about CO₂ mineral carbonation processes. The reaction scheme can be summarized as follows:

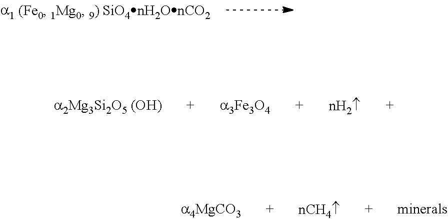

In the above scheme, α₁--->α₄ are the stoichiometric coefficients of the phases that participate in the process.

During this process, it is thus possible to observe the mineralization of CO₂ as a solid carbonate as well as its transformation into methane and other low molecular weight hydrocarbons, which is made possible by the presence of molecular hydrogen (H₂) produced by the dissociation of H₂O occurring during the first step of the process. It should be noted that such processes already occur in nature, mainly in ocean floors, although with extremely slow reaction kinetics on a geological time scale, and have also been reproduced in the laboratory, although in the presence of particularly high pressure and temperature conditions.^{[10]} Along with the specific case of Olivine, silicates of alkaline earth metals (e.g., Ca and Mg) and transition metals (e.g., Fe) are widely present in nature as constituents of various minerals and rocky materials, while others are obtained as by-products of industrial manufacturing processes.

Such classes of products include, for example, basalt rocks, i.e. rocks formed by rapid solidification of lava produced by volcanic eruptions in contact with air or water, as well as some types of solid residues from steelmaking processes. The chemical composition and the crystallographic characteristics of the two above-mentioned classes of materials have some elements in common and have aroused interest in relation to the CO₂ transformation process. The following will briefly describe some details concerning the reactivity and the transformations observed for each one of the two above-mentioned classes of products.
1) The world production of iron and steel has shown a rapid growth in the last twenty years. From 2000 to 2020, the production of raw steel has increased from 850 to 1,878 millions of tons. Steel is produced industrially mostly through the use of two distinct production processes: the integral cycle, which uses raw materials like iron mineral and pit coal, and the electric arc furnace cycle, which melts ferrous scraps and exploits the characteristics of complete recyclability of steel, which cycles are then followed by various refinement processes. In addition to the primary product, i.e. steel, the integral cycle and the electric arc furnace cycle (EAF) produce also other materials, such as the so-called slags. The latter have variable characteristics and peculiarities which depend on many factors, ranging from the raw materials employed to the production technologies adopted. For example, slags deriving from steelmaking processes can be divided into four main classes: (i) blast furnace slags (or blast furnace dross); (ii) oxygen converter (BOF, Basic Oxygen Steelmaking) slags; (iii) electric arc furnace (EAF) slags; (iv) secondary metallurgy slags. EAF and BOF slags are produced in larger amounts, being respectively 28% and 70% of the total quantity, and have mineralogical and compositional characteristics that are not very dissimilar, although they are originated by different production processes. In particular, the so-called black slags formed during the EAF metallurgy process (melting in electric arc furnace) executed on ferrous and cast-iron scraps by supplying heat in a basic environment (obtained by adding lime or limestone), mainly consist of calcium oxide (CaO), magnesium oxide (MgO), iron oxides (FeO), quartz (SiO₂), and silicates containing metals in concentrations that depend on the starting raw materials employed, the type of steel produced, and other specific process conditions. Such materials are produced in very large quantities: as regards Italy alone, the amount of such by-products is approximately 3 Mt/y, estimating that 100-150 kg of slags are produced per ton of steel. Hence the interest shown by engineers in exploiting such products, which, depending on their specific properties, are now either discarded or used in the building industry as inert materials. Alongside well-known applications that have now reached technological maturity, it must be pointed out that a number of papers have recently been published in relation to the potential use of such materials in CO₂ conversion processes under known traditional conditions, i.e., by thermal activation. Based on the nature of the just above-described slag, in fact, under particular temperature and pressure conditions, it can react with CO₂, resulting in carbonate formation. This process is called mineral carbonation, and simulates the natural ageing of rocks that occurs in nature, albeit with very slow kinetics. More specifically, carbon dioxide in gaseous form can react with the silicate that constitutes the residue, forming the corresponding carbonate phase. Thus, CO₂ can be stored permanently in solid form according to the following scheme, wherein Ca²⁺ is represented as a metal ion:

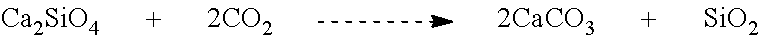

2) Basalt is the main constituent of the upper part of the ocean crust, and large basalt deposits can be found in many regions of the world. The main mineral components of basalt are calcium plagioclase, pyroxenes, and sometimes olivine. Such components belong, from a crystallographic viewpoint, to different silicate families. Thanks to its mechanical and morphological properties, basalt finds numerous applications in the building industry (elements for floors, coverings, draining systems, road edges, and so forth), for ornamental and decorative elements (statues, chimneys, urban decoration, and so forth), and also in agriculture, e.g. as plant fortifier. The extended industrial utilization as a building material results in the production of large volumes of slags and scraps of different sizes and granulometry, which in turn are either reused in the building industry as filling materials or discarded. A study campaign has recently been started for evaluating it as a material for geological storage of CO₂, while the chemical reactivity of basalt is the one that is typical of the various Ca, Mg, Fe, etc. silicates, which can give carbonation processes in accordance with the above-discussed classical schemes.

The great amount of materials belonging to such classes, produced as scraps of industrial processes, and their reactivity with CO₂, are the reasons why the industry is showing great interest in setting up processes for large-scale exploitation of such materials. To the present inventors' knowledge, for both of the above-mentioned classes of products no literature data are available which concern the use of such materials in mechanically activated processes under the conditions specified in the present description.

Considering the conditions required for the activation of such process by thermal means, the use of less conventional forms of energy such as, for example, kinetic energy, may offer some specific solutions of interest. In particular, the study of the transformations induced in chemical systems comprising at least one solid phase by supplying mechanical energy is generally referred to as “mechanochemistry”: such transformations are achieved through the use of particular apparatuses, i.e. the so-called mechanochemical reactors, which may have different construction specifications and perform different mechanical actions. For example, high-energy micromills or rotary mills provided with milling means (such as balls made of suitable materials and having variable sizes) are examples of apparatuses that are commonly employed, on a laboratory scale, for the execution of mechanochemical processes. However, other types of devices provided with suitable milling means can be used as well for executing mechanochemical processes. The continuous repetition of processes of breaking and comminution of the solid phases involved, the renewal of the exposed surfaces of the mechanically processed phases, and the local conditions achieved often make it possible to successfully obtain chemical transformations that could not otherwise be obtained under traditional thermal treatment conditions. The versatility of these mechanical processes and the specific conditions that can be obtained via interaction between solid phases and gaseous phases, as well as the industrial proliferation of processes involving solid phases, support the study of this mode of activation of mechanochemical processes for CO₂ conversion, for which, however, scientific literature data are still very scant and lack a global vision that may clarify their feasibility, specificity and possible application advantages.

As already highlighted above, in order to contribute to mitigating climate changes by reducing CO₂ emissions, the Carbon Capture Storage (CCS) and the more recent Carbon Capture Utilization (CCU) strategies have drawn the attention of scientists all over the world, as well of industry. Among theese, mineral carbonation, also known as “mineral sequestration”, is the most renowned and widespread CO₂ storage technique. The main advantage of mineral carbonation is the formation of stable carbonates capable of “storing” CO₂ for long periods, thanks to its conversion into carbonate trough reaction with metal oxides. It is within this frame that several studies have been developed in an attempt to exploit the interaction between black slags and CO₂.

Carbonation processes can be divided into two broad categories: i) Indirect carbonation, ii) Direct carbonation. In the indirect carbonation process i), the reactive phase is initially extracted from the mineral, and then the intermediate products are carbonated. In the direct carbonation process ii), the reaction between alkaline solids and CO₂ in gaseous form occurs in a single stage. The advantage of this process lies in its simplicity and minimal use of chemical reagents. Direct methods are in turn subdivided into two categories: iia) Gas-solid carbonation, which is believed to be the simplest approach, wherein alkaline solid residues are made to react with gaseous CO₂ under appropriate temperature and pressure conditions; and iib) Aqueous-phase carbonation, wherein the process occurs in a single stage that involves direct CO₂ sequestration in a suspension of alkaline slags (slurry, thin film, or aqueous solution). [J. Wang, (2021), A Review of the Application of Steel Slag in CO₂ Fixation. ChemBioEng Reviews, 8(3), 189-199; R. Ragipani et al. (2021), A review on steel slag valorization via mineral carbonation. Reaction Chemistry & Engineering, 6(7), 1152-1178]. In this contest, the CO₂ storage process using industrial solid residues, such as steelmaking slags, has been studied experimentally by using indirect methods [S. Teir et al. (2007), Dissolution of steelmaking slags in acetic acid for precipitated calcium carbonate production. Energy, 32(4), 528-539; Hall, C. Hall et al. (2014), Calcium leaching from waste steelmaking slag: Significance of leachate chemistry and effects on slag grain mineralogy. Minerals Engineering, 65, 156-162; R. Ragipani et al. (2020), Understanding dissolution characteristics of steel slag for resource recovery. Waste Management, 117, 179-187], gas-solid direct methods [J. Yu et al. (2011), Study on characteristics of steel slag for CO₂ capture. Energy & fuels, 25(11), 5483-5492; T. D. Rushendra Revathy, (2016), Direct mineral carbonation of steelmaking slag for CO₂ sequestration at room temperature. Environmental Science and Pollution Research, 23(8), 7349-7359], and aqueous-solution direct methods [E. E. Chang et al. (2011), Performance evaluation for carbonation of steel-making slags in a slurry reactor. Journal of hazardous materials, 186(1), 558-564; R. Baciocchi et al. (2011), Wet versus slurry carbonation of EAF steel slag. Greenhouse Gases: Science and Technology, 1(4), 312-319; S. N. Lekakh et al. (2008), Kinetics of aqueous leaching and carbonization of steelmaking slag. Metallurgical and Materials Transactions B, 39(1), 125-134]. In the above-mentioned experimental works, no reference is made to mechanical activation techniques in carbonation processes using steelmaking slags.

Also, as concerns the application aspects of the interaction between steelmaking slags and CO₂, it is necessary to mention Chinese patent no. CN112791573A entitled “Steelmaking waste collaborative treatment system and method based on ball milling strengthening”, which generally combines mechanical activation processes with steelmaking slags for possibly capturing CO₂, among other things, under conditions that are, however, totally different from those of the present invention. Moreover, the chemical aspects of the process are not discussed.

The literature data currently available about the interaction between CO₂ and basalt concern geological storage and possibly processes induced by thermal or hydro-thermal treatment [Kelemen P. et al., An Overview of the Status and Challenges of CO₂ Storage in Minerals and Geological Formations. Frontiers in Climate, 2019, 1, 9; Zhang, L., et al., Geochemistry in geologic CO₂ utilization and storage: A brief review. Advances in Geo-Energy Research, 2019, 3, 304-313; Gislason S. R. et al., Geochemistry: Carbon Storage in Basalt. Science 2014, Vol 344; Sanna A. et al., A review of mineral carbonation technologies to sequester CO₂. 2014, Chem. Soc. Rev., 43, 8049-8080; Sissmann O. et al., Enhanced Olivine Carbonation within a Basalt as Compared to Single-Phase Experiments: Re-evaluating the Potential of CO₂ Mineral Sequestration. Environ. Sci. Technol. 2014, 48, 5512-5519; Mattera J. M. et al., Permanent Carbon Dioxide Storage into Basalt: The CarbFix Pilot Project, Iceland. 2009, Energy Procedia 1, 3641-3646; Daval D. et al., Carbonation of Ca-bearing silicates, the case of wollastonite: Experimental investigations and kinetic modelling. Chemical Geology, 2009, 262, 262-277; Xiong W. et al., CO₂ Mineral Sequestration in Naturally Porous Basalt. Environ. Sci. Technol. Lett. 2018, 5, 142-147]; lastly, the work by Rigopoulos G. et al., Carbon dioxide storage in olivine basalts: Effect of ball milling process, Powder Technology. 2015, 273, 220-229, evaluates mechanical activation processes using basalt powders for CO₂ interaction under conditions that are, however, totally different from those of the present invention.

### Drawbacks of the Prior Art

Notwithstanding the importance of the above-illustrated themes from an environmental and economical viewpoint, to the present inventors' knowledge no consolidated and commercially available technologies currently exist which are applicable at an industrial level for controlling the processes of capturing, storing and converting CO₂ released into the atmosphere. The intense research activity of the scientific community and of the skilled technician in this field, referable to the above-mentioned CCS (Carbon Capture and Storage) and CCU (Carbon Capture and Utilization) processes, has been mostly directed to studying, respectively, mineral carbonation processes and confinement on geological sites and conversion processes via high-temperature catalytic treatments. In this latter case, the focus is mainly on setting up complex catalytic systems, often obtained starting from precious metals and other chemical systems, via procedures referred to as fine chemistry, and such catalysts generally cannot be easily and economically produced and used on an industrial scale. Furthermore, the operating conditions required by the conversion processes, even in the presence of the above-mentioned catalytic systems, are far from standard optimal conditions: the temperatures of the processes for transforming CO₂ into fuels, studied under pre-application conditions, are generally >300° C. and represent themselves a source of high energy consumption and environmental pollution.

Lastly, it must be underlined that no technologies are currently available which are based on the transfer of mechanical energy for activating the above-mentioned CO₂ capture, storage and utilization processes, and which are applicable on an industrial scale.

Another aspect must also be pointed out, which has not been taken into account in previous studies while however being connected with the experimental/actual conditions under which CO₂ is released into the atmosphere. When not related to natural phenomena, the processes of CO₂ production are connected with processes of combustion of organic materials used on an industrial scale, generally fossil fuels (but this also applies to combustion of biomass, wood, coal, etc.), such as, for example, energy production processes in thermoelectric power plants, iron and cement production, processes related to automotive systems, as well as air-conditioning processes, feeding processes, etc. The experimental conditions under which the combustion phenomena occur are far from the standard ones (e.g., the combustion of fossil materials in the boilers of thermoelectric power plants reaches temperature values (T) of approximately 560° C., i.e. much higher than ambient temperature, and pressure values (P) much higher than atmospheric pressure), but the produced flow of CO₂ is generally released into the atmosphere at a pressure only slightly higher than atmospheric pressure and at a temperature of approximately 100° C. Merely by way of non-limiting example, the exhausted fumes exiting post-combustion chimneys in the typical conditions of a thermoelectric power plant are characterized by temperature values T of approximately 100° C. (90-95° C.) and a pressure P close to atmospheric pressure (approx. 100,000 Pa). Moreover, their composition generally includes N₂ (approx. 75%), O₂ (approx. 9%), H₂O (approx. 6%), CO₂ (approx. 10%). Therefore, the fumes consist of a humid mixture containing excess thermal energy and kinetic energy, and are released into the atmosphere under continuous flow conditions, at a velocity of 10 to 20 m/s, and with flow rate values that depend on the size of the chimney. Conditions not too different from these can be found in other industrial chimneys and in downstream exhaust systems of other processes of combustion of fossil materials.

### Technical Problem

In light of the above discussion, it has become particularly important to realize novel process of CO₂ conversion, induced by mechanical activation under gas flow conditions, in particular as a gas mixture having a CO₂ content (preferably, approx. 8-10% by volume) comparable to the above-mentioned data concerning industrial exhaust fumes, and in continuous mode, so that it can advantageously be applied to, for example, said exhaust fumes produced by industrial processes, where a batch mode would not be profitably applicable.

Therefore, the need is still strongly felt by those skilled in the art for an economical and environmentally compatible method of, for example, converting carbon dioxide (CO₂), whether alone or possibly mixed with other gases, into high added value chemical compounds under continuous gas flow conditions.

The object of the present invention is to provide an adequate solution to the above-highlighted technical problem.

### Description of the Invention

The present inventors' attention has been focused on processes for the production of hydrogen (H₂) from water (which thus acts as a reactant, not as a solvent) and for CO₂ conversion induced by mechanical activation in the presence of slags from industrial processes, particularly slags from iron and steel production processes and basalt processing scraps. Such slags unexpectedly showed to be materials particularly suitable for activating mechanically induced CO₂ conversion processes, although they had always been considered, from a theoretical viewpoint, to be chemical systems having low economical value and characterized by poor chemical reactivity.

Solid samples of such materials were used as reactants in a number of tests aimed at evaluating CO₂ conversion. The solid reactants showed high reactivity characterized by very high CO₂ conversion values and extremely fast kinetic profiles: in particular, conversion values reached 100% in tests carried out on steelmaking slags, and remained stable for long treatment times.

This research highlighted the dependency on the experimental conditions in use. The application potentialities unexpectedly turned out to be very interesting due to the reactivity that was observed, the chemical characteristics of the materials, which were studied with a view to circular economy, and the easy process scale-up even under conditions wherein the kinetic energy of the combustion gas is transformed into mechanical energy for activating the mechanochemical reactor.

Particular attention was paid to studying/optimizing the experimental conditions of the process, so as to make them compatible with the implementation conditions of several industrial-scale processes producing large amounts of CO₂. The present invention relates to a chemical process, conducted by mechanical activation (i.e., a mechanochemical process) under continuous gas flow conditions, for converting CO₂ into a mixture of high added value chemical compounds comprising a mixture of low molecular weight hydrocarbons, preferably including mainly methane, ethylene and ethane, along with solid products of mineral carbonation, e.g., Ca, Mg and Fe carbonates. Therefore, CO₂ conversion is effected by means of a continuous mechanochemical process under gas flow, in the absence of any non-aqueous solvents (it should be considered that any water that may be present is actually a reactant that produces hydrogen; therefore, it is not a solvent in this case), and starting from reactants consisting of powders of industrial slags, such as, respectively, steelmaking slags (e.g. EAF, Electric Arc Furnace, slags) and basalt processing scraps (both materials containing different phases of Mg, Fe and Ca silicates), H₂O (deionized or not), and CO₂ as the only component in the gaseous phase, or else a gas flow containing CO₂ in amounts similar to those previously specified herein for exhaust fumes released from post-combustion chimneys of an industrial manufacturing process or the like. Said amount of CO₂ in said gas flow is preferably in the range of approx. 4% to 20%; more preferably, 5% to 18%; more preferably, 6% to 15%; even more preferably, 7% to 13%; even more preferably, 8% to 10%. The solid reactants are inserted into a suitable mechanochemical reactor constructed (by way of non-limiting example) starting from a ball mill driven by, for example, an electric motor (other driving means may however be used, as will be specified hereinafter). The solid phases are subjected to mechanical treatment by suitable milling bodies (e.g., balls made of steel or other suitable materials, or other milling means adequate for the type of reactor in use), and are put under conditions of reaction with the fluidic reactants (CO₂, H₂O, or a flow of humid gases containing CO₂ in the above-specified amounts), which in turn are transported into and through the reactor under continuous flow conditions.

The process is characterized by the absence of any non-aqueous solvents (as previously explained in detail, water is, in this case, an actual reactant and does not act as a solvent; therefore, it can be appropriately stated that the process occurs in the absence of any solvents), by the method of activation of the process (using mechanical energy supplied by the milling bodies to the reactants that are trapped during ball-ball or ball-jar collisions), by very high yield values of the CO₂ transformation process, and by very fast reaction kinetics, in addition to rather mild process conditions (e.g. temperature (T) and pressure (P)) comparable to those described above with reference to exhaust fumes released from post-combustion chimneys of industrial manufacturing processes or processes of combustion of organic materials (e.g. combustion of wood, coal, oil, petrol, diesel fuel, gas and/or biomass).

Such characteristics lend environmental sustainability properties to the process of the invention, resulting in great potential interest in the commercial exploitation of said process.

The mechanism through which the series of chemical transformations of the process occur is certainly complex and comprises several stages, including dissociation of the H₂O molecule (water being thus a reactant), dissociation of the CO₂ molecule, surface adsorption processes, chemical absorption processes, bulk scattering, structural evolution of the solid phases, synthesis of hydrocarbons, processes of desorption of gaseous phases from the surfaces of the solids.

A detailed evaluation of such aspects goes beyond the application properties related to the scope of the present invention; it is however of interest here to underline that during the initial phases of the process, depending on the reactants in use, large amounts of H₂, i.e., hydrogen in molecular form (with reference to the reaction under examination), are produced, originated from the dissociation of water promoted by the kinetic energy derived from the impact of the milling bodies against the humid powders of the solid reactants. Formation of H₂ is also correlated with processes of oxidation (of Fe²⁺ to give Fe³⁺) that can be observed in the different solid phases under examination.

Once hydrogen has been produced, in the presence of CO₂, it shows to be able to induce carbon dioxide reduction, again thanks to the mechanical energy generated and transferred during the non-elastic collisions occurring between the milling bodies, the powders of the solid scraps under examination, and the reactants both in liquid and gaseous phase.

In summary, it is the object of the present invention a continuous mechanochemical process under gas flow to carry out the conversion of CO₂ into a mixture of high added value chemical compounds, said mixture comprising a mixture of low molecular weight hydrocarbons, preferably including mainly methane, ethylene and ethane, and solid products of mineral carbonation, preferably Ca, Mg and Fe carbonates, said process comprising at least the following steps:
a)—continuously passing a gas flow comprising, or consisting of, CO₂ through a reactor equipped with milling means, and in the presence of suitable solid reactants in powder form, belonging to the previously mentioned classes of industrial processing slags, and water, in liquid or gaseous form, at a temperature from room temperature (approx. 18-30° C., preferably 20-25° C.) to ≤100° C.; said reactor being appropriately subjected to motion in such a way that the milling means trigger a mechanochemical reaction that, on one side, produces hydrogen from the present water, and then said hydrogen thus produced carries out the conversion of a portion of CO₂ into said low molecular weight hydrocarbons, while, on the other side, another portion of CO₂ reacts with the transformation products of the silicates contained in the solid reactants under examination, mentioned above, and with the non-reacted H₂O to give said products of mineral carbonation as previously described herein;
b)—separating and recovering from the gas mixture exiting the reactor said low molecular weight hydrocarbons and non-reacted hydrogen obtained in step a);
c)—at the end of the reaction, separating and recovering from the reactor said solid products of mineral carbonation of the silicates contained in the reacting solid materials, obtained in step a).

The advantages of the technique (process) of the present invention, as described above, include at least the following:
A) use of a low-cost natural mineral (slags from industrial processes, in particular, for example, basalt processing scraps and black slags obtained from EAF steelmaking processes; estimated price: approx. 10 USD/ton);
B) generation of the hydrogen necessary for the process of chemical reduction of carbon dioxide into methane according to the following reaction scheme:

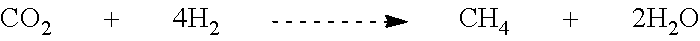

directly from the water contained in the gas mixture;
C) execution of the said conversion process at a low temperature (from room temperature to approx. ≤100° C.; preferably, to approx. ≤80° C.; more preferably, to approx. ≤60° C. or ≤40° C.);
D) further use of the solid materials thus obtained, which, once exhausted, are inert in terms of toxicity and chemical noxiousness, and any metals that may be present therein can be extracted using methods known in the art; they are also totally reusable in the cement industry (e.g., to give magnesia cement), thus not representing a problem for the environment;
E) production of energy vectors (hydrogen and methane and light hydrocarbons, e.g., ethane and ethylene) and construction materials (magnesium oxide-magnesia cement).

### BRIEF DESCRIPTION OF THE FIGURES

FIG. 1 shows an illustrative, but merely non-limiting, embodiment of the experimental apparatus of the present invention.

FIG. 2 represents the relationship between conversion (X), selectivity (S) and yield (Y) when products B and C are obtained from the reactants, indicated as A, plus a certain quantity of non-reacted reactant A. At time t1 the process has not yet begun, and only the reactants (A) are present. At time t2, following the start of the process, the system will be composed of the products (B and C) and a part of reactants (A) not yet reacted.

FIG. 3 shows the conversion rate (%) of CO₂ during the preliminary tests conducted on EAF slags under batch conditions.

FIG. 4 shows the trend of pressure within the mechanochemical reactor, as a function of milling time, during the preliminary test conducted on EAF slags under batch conditions.

FIG. 5 shows the results of the XRD analysis conducted on slag samples from EAF processes subjected to the preliminary tests with the mechanochemical reactor under batch conditions. The lower curve refers to the raw sample, the middle curve refers to the sample treated for 40 minutes, and the upper curve refers to the sample treated for 120 minutes under the specified conditions.

FIG. 6 shows the kinetics of the CO₂ conversion process on EAF slags under continuous reactor feeding conditions.

FIG. 7 shows the CO₂ conversion rate (%) as a function of mechanical treatment time. In the box: Chromatographic gas analysis of the mixture of reaction gases in samples taken at the beginning of the process and after 40 minutes of mechanical treatment, respectively.

FIG. 8 shows the X-ray diffraction pattern and the semiquantitative data analysis conducted with the Rietveld method, concerning samples of slags from EAF processes at the beginning and at the end of the 180-minute period of mechanical treatment under the conditions specified in the description.

FIG. 9 shows the IR spectra of, respectively, the unprocessed EAF slag sample (initial sample, black line) and the sample picked up after 180 minutes of mechanical treatment (red-brown line).

FIG. 10 shows the kinetic trend of CO₂ conversion in tests conducted on slags from EAF processes, with different values of gaseous mixture flow rate.

FIG. 11 shows a comparison between the kinetic curves concerning tests conducted on slags from EAF processes under different mechanical activation conditions. The black line refers to the test conducted using 3 balls having a mass of 4 g each at a motor revolution speed of 875 RPM. The blue line refers to the test conducted using 5 balls having a mass of 0.4 g each at a motor revolution speed of 745 RPM.

FIG. 12 shows the kinetic trends, expressed as the number of converted CO₂ moles as a function of time, observed in tests conducted on slags from EAF processes subjected to mechanical treatment in atmospheres consisting of gaseous mixtures characterized by different CO₂ percent contents (respectively, 10%, 25%, 50%), as indicated in the Figure.

FIG. 13 shows the kinetic curves, expressed as percentage of converted CO₂ as a function of time, observed in tests conducted on slags from EAF processes subjected to mechanical treatment in atmosphere consisting of gaseous mixtures characterized by different CO₂ percent contents, as indicated in the Figure.

FIG. 14 shows the kinetic curve, expressed as percentage of converted CO₂ as a function of time, observed in a test conducted with no steel milling bodies, but with 3 residues of slags from EAF processes having a mass of 1 g each.

FIG. 15A shows the kinetic curves, expressed as percentage of the quantity of converted CO₂ as a function of time, observed in tests conducted on basalt powders produced during different industrial processing stages.

FIG. 15B shows the kinetic curves, expressed as % concentration (v/v) of generated H₂ as a function of time.

FIG. 16 shows the kinetic curves of the CO₂ conversion process in processes conducted on basalt powders under different mechanical treatment conditions.

FIG. 17A shows the kinetic curves related to the CO₂ conversion process;

FIG. 17B shows the trend of H₂ concentration over time;

FIG. 17C shows the trend of CH₄ concentration over time. The data in the above FIGS. 17A-17C refer to the atmosphere of the mechanochemical reactor in processes conducted on basalt powders under different conditions of flow of the reacting gaseous phase.

FIG. 18 shows the XRD analysis of basalt powders: alongside the experimental data in dot form, it shows the fitting obtained using the Rietveld method, as well as the profiles relating to the different crystallographic phases. The lower profile (0 min) refers to basalt powders pre-ground for 1 h in non-reactive atmosphere, whereas the upper profile refers to a sample analysed at the end of the mechanochemical process in the presence of CO₂.

FIGS. 19A and 19B show the FT-IR spectra of basalt powders subjected to CO₂ conversion tests under different mechanical treatment conditions.

FIG. 20 shows the kinetic curves of the CO₂ conversion process and of the H₂ and CH₄ concentration values in the atmosphere of the mechanochemical reactor in processes conducted on basalt powders using different compositions of the reacting gaseous mixture.

FIG. 21 shows the X-ray diffraction analysis of the EAF/Fe(10%) composite system.

FIG. 22 shows the X-ray diffraction analysis of the EAF/Ca(10%) composite system.

FIG. 23 shows the X-ray diffraction analysis of the EAF/Mg(10%) composite system.

FIG. 24 shows the kinetics of the CO₂ conversion process conducted on the EAF/Fe(10%) system.

FIG. 25 shows the kinetics of the CO₂ conversion process conducted on the EAF/Ca(10%) system.

FIG. 26 shows the kinetic trend of H₂ evolution in the CO₂ conversion process conducted on the EAF/Ca(10%) system.

FIG. 27 shows the kinetics of the CO₂ conversion process conducted on the EAF/Mg(10%) system.

FIG. 28 shows the kinetic trend of H₂ evolution in the CO₂ conversion process conducted on the EAF/Mg(10%) system.

FIG. 29 shows the kinetic trend of CH₄ evolution in the CO₂ conversion process conducted on the EAF/Mg(10%) system.

FIG. 30 shows the FT-IR spectra observed on samples of the three solid systems (respectively, EAF/Mg(10%), EAF/Ca(10%), EAF/Fe(10%)) at the end of the mechanochemical tests.

### EXPERIMENTAL SECTION

### Description of the Reactor and Reaction Conditions

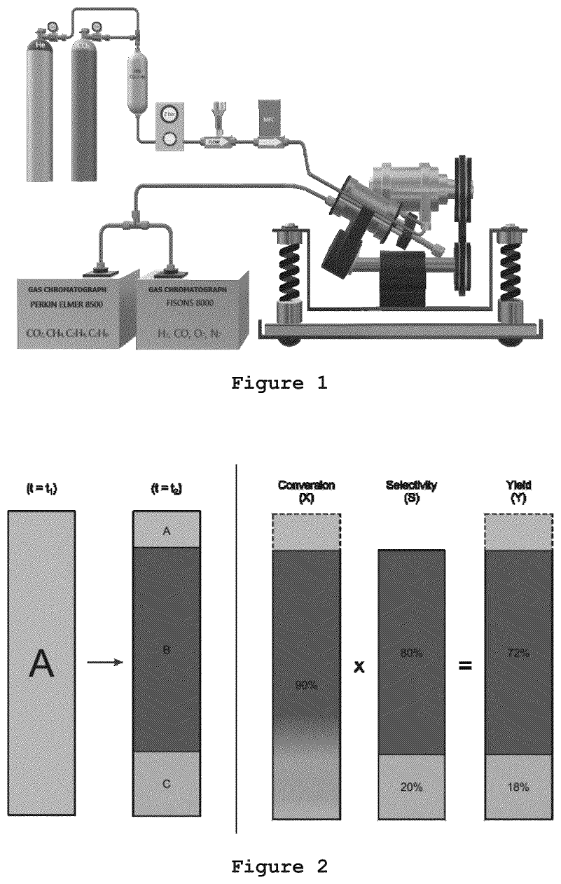

The following experimental section will describe, by way of illustrative example, some of the characteristic aspects of the present invention, without however by no means limiting the broad application potential thereof. Based on the following explanations, those skilled in the art will have no difficulty in applying and modifying the teachings provided herein by adapting the dimensions, quantities and construction aspects to specific practical implementations. In one embodiment, the equipment used for the experimental activity is schematized in the annexed FIG. 1. In this case, it essentially consists of a cylindrical reactor (e.g. a grinding jar (mill) having an internal volume of approx. 65 ml), suitable for the execution of mechanical treatments by means of ball milling processes. The reactor and the milling bodies are made of hardened stainless steel (of course, the dimensions and materials of the reactor may be freely selected and/or defined for specific applications, depending on the type and rate of gas flow (e.g. an exhaust gas) to be treated. The same also applies to the number, size and material of the milling means/bodies). The cylindrical reactor is equipped with gas-tight covers and gas transfer lines. The gas lines, e.g., partly made of steel and partly consisting of flexible Teflon (PTFE) hoses, provide the connection between the reactant gas supply (or the exhaust gas flow) and the reactor, and between the latter and two gas chromatographs for analysing the reactant and the produced gases. Unless otherwise specified, such a “gas flow” configuration of the reactor permitted the execution of tests under a continuous supply of gaseous reactants. The gas chromatographs are both equipped with detectors suitable for a quantitative evaluation of the gaseous phases of interest. A first gas chromatograph, equipped with a GS-Q wide-bore capillary column and TCD-FID detectors, with He as a carrier gas, made it possible to evaluate the CO₂ through the TCD detector and the low molecular weight hydrocarbons through the FID detector. A second gas chromatograph, equipped with a column packed with 13× (10Λ) molecular sieves and a TCD detector, with Ar as a carrier gas, made it possible to evaluate the amounts of H₂, CO, O₂ and N₂.

The mechanochemical reactor was housed in the seat of a known mill Spex Mixer/Mill mod.8000 [Spex Certiprep., https://www(dot)spexcertiprep(dot)com], suitably modified and equipped with a three-phase electric motor controlled by a speed regulator, which provides control over the number of revolutions of the motor shaft and imparts to the mill a three-dimensional motion in space. The tests were conducted under two different conditions: at 875 and 1,000 revolutions per minute (rpm) of the motor shaft, respectively corresponding to 14.5 and 16.6 Hz.

In practice, the revolution speed of the reactor can be varied, depending on the type of application, from 500 rpm to 1,500 rpm; preferably, from 600 rpm to 1,400 rpm; more preferably, from 700 rpm to 1,300 rpm; even more preferably, from 800 rpm to 1,200 rpm.

The tests were conducted using solid slag samples having a mass of 2 g of EAF slags derived from processes for the production of ferrous materials, or 2 g of basalt processing slags.

The above-described conditions correspond to a solid reactant powders (slags):H₂O stoichiometric ratio of 1:2.

The reactor was connected to the gas lines and housed in the support of the Spex 8000 commercial mill, for the execution of the reactive tests.

The gaseous reaction mixture was composed of CO₂ (Sapio, purity of 99.995), with abundance (percent quantity) of 10% by volume in He or N₂ (Sapio, BIP purity). After saturating the atmosphere of the mechanochemical reactor, the reactive mixture was fed in a flow of 5 ml/min in the tests conducted on black slag powders from EAF processes, and 1 ml/min in the tests conducted on basalt processing scrap powders. The flow was kept constant through a regulation valve and measured with a mass flowmeter (in the applications of the process of the invention, the flow rate may be changed freely according to the amount of CO₂-containing exhaust fumes released per time unit, the chimney size, and other parameters related to the release of said fumes). The composition of the reactive mixture gases for the tests was selected in tight relation with the average composition data of the mixture of CO₂-containing fumes exiting the previously mentioned industrial combustion processes (see above).

The structural analysis of the powders of unprocessed samples and of the samples subjected to mechanical treatments for selected treatment times was carried out by X-ray diffraction (XRD) using a Rigaku SmartLab diffractometer with a rotating anode source (λ_{Cu}=1.54178 Å) and equipped with a graphite monochromator on the diffracted beam, employing a Bragg-Brentano geometry. The identification of the different phases in the diffractograms and the evaluation of their relative abundance and of some microstructural parameters were conducted using the Rietveld microstructural refinement method.

Pre-reaction and post-reaction samples were also characterized by infrared spectroscopy (FT-IR) using a Jasco FT-IR 4600 instrument in ATR mode.

### Description of the Results of the CO₂ Conversion Tests and of the Analyses of the Solid and Gaseous Phases

The following will present the results of the CO₂ conversion tests under the above-described conditions. Data will be provided about the analyses of the gaseous phases conducted by gas chromatography, and about the results of the analyses of the solid phases conducted by X-ray diffraction techniques and by FT-IR spectroscopy.

The mechanical treatment in reactive atmosphere was conducted using 3 steel balls having a mass of 4 g each and by setting the revolution speed of the motor to 875 rpm, unless otherwise specified. The kinetics of the CO₂ conversion process was monitored by evaluating the degree of CO₂ conversion via measurement of the concentration of CO₂ eluted from the reactor after selected mechanical treatment times up to 180 minutes.

### Definition of the Descriptor Parameters of the Process

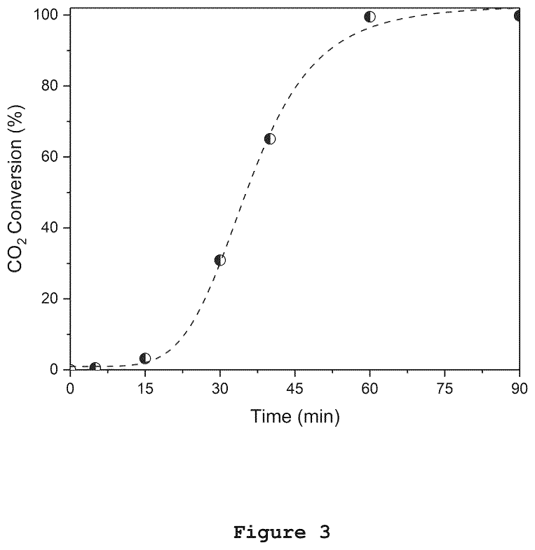

The study of the process was conducted by monitoring, as parameters, the rate of CO₂ “conversion”, the “selectivity” in obtaining the products, and the “yield” of a specific product. The annexed FIG. 2 illustrates, in graphic form, the meaning of these three descriptor parameters.

### CO₂ Percent Conversion

It indicates the CO₂ transformation rate (%) in comparison with the initial concentration, and is expressed by the following mathematical relation:

$${CO}_{2} conversion , \% = \frac{{[ {CO}_{2} ]}_{i} - {[ {CO}_{2} ]}_{t}}{{[ {CO}_{2} ]}_{i}} \cdot 100$$

where “[CO₂]ᵢ” means the CO₂ concentration at time “i=initial”, i.e., before the start of the process; and
where “[CO₂]ₜ” means the CO₂ concentration at time “t”, i.e., at a time instant after the start of the process.

In gas chromatography, the concentration of an analyte can be expressed as the product of a response factor and the area of the signal of the analyte under evaluation. From a mathematical viewpoint, this can be expressed as:

$$[ {CO}_{2} ] = {f}_{r} \cdot Area of {CO}_{2} signal$$

where “[CO_{2]}” is the concentration of the carbon dioxide analyte; and
where “fᵣ” is the response factor for the CO₂ analyte and refers to a specfic column and a specfic method of analysis.

Therefore, with appropriate substitutions and simplifications, the equation for calculating the percent CO₂ conversion rate can be rewritten as:

$$CO 2 conversion , \% = \frac{( {f}_{r} \cdot Area {CO}_{{2}_{i}} ) - ( {f}_{r} \cdot Area {CO}_{{2}_{t}} )}{( {f}_{r} \cdot Area {CO}_{{2}_{i}} )} \cdot 100$$

$$CO 2 conversion , \% = \frac{{f}_{r} \cdot ( Area {CO}_{{2}_{i}} - Area {CO}_{{2}_{t}} )}{{f}_{r} \cdot Area {CO}_{{2}_{i}}} \cdot 100$$

$${CO}_{2} conversion , \% = \frac{Area {CO}_{{2}_{i}} - Area {CO}_{{2}_{t}}}{Area {CO}_{{2}_{i}}} \cdot 10 \; 0 .$$

### Selectivity

It expresses the ratio between the concentration of a specific product and the total concentration of the products. It can be calculated by means of the following mathematical relation (expressed here for methane, by way of example):

$${CH}_{4} Selectivity , \% = \frac{[ {CH}_{4} ]}{[ X ]} \cdot 100 ,$$

$$where :$$

$$[ X ] = total concentration of the products .$$

This relation is applied in order to obtain the selectivity of the detectable products, taking into account that, for each one of them, it will be necessary to draw a calibration straight line in order to be able to obtain the concentration.

Yield of the Reaction Giving Methane (and/or Other Products)

It expresses the percent abundance (quantity) of a product relative to the conversion rate of the process. From a mathematical viewpoint, it can be expressed as the product of selectivity and conversion:

$${CH}_{4} Yield , \% = {CH}_{4} Selectivity - {CO}_{2} Conversion / 100$$

### Results

The main innovative aspects of the present patent application lie in the chemical nature of the solid-phase reactants which have been, or can be, used, and in the reactivity shown by such reactants in the CO₂ conversion process by mechanical activation. In particular, the present inventors focused their research activity, without limitation, on the following two classes of materials obtained from industrial waste:
1) slags derived/obtained from metallurgical processes for the production of ferrous materials, e.g., those known in the literature as EAF slags; and/or
2) residues derived/obtained from basalt processing activities.

For such classes of materials, to the present inventors' knowledge, significant literature data are very scant (substantially null) in regard to the processes discussed herein, and the results obtained under the experimental conditions described herein represent an absolute novelty.

The chemical composition and the crystallographic characteristics of the two above-mentioned classes of reactant materials have some elements in common, and both lead, during the CO₂ transformation process, mainly to the formation of “carbonate” phases and, when using basalt scraps, also to the production of light hydrocarbons in the gaseous phase.

The following will describe some details concerning the reactivity and the transformations observed for each class involved in the present invention.

1) Results of the CO₂ Conversion Process Conducted on “Black Slags” Derived/Obtained from Metallurgical Processes for the Production of Ferrous Materials (EAF).

The present study was developed starting from the considerations set out in the previous sections, with a view to exploiting slags coming from the EAF metallurgical process, which materials are now commonly available at low costs, in the process of converting CO₂ activated by mechanical means. The first tests conducted on such materials were directed to evaluating the reactivity towards CO₂; to such end, the mechanochemical reactor described in the previous section was used as a batch-type reactor. This means that the reactor, into which 2 g of EAF slag powders and 0.3 ml of H₂O were introduced, was pressurized, after purging the gaseous atmosphere, with CO₂ to 2.5 bar (1.5 bar above atmospheric pressure). The reactor was then set in motion to 875 RPM, and several tests were carried out with increasing milling times, at the end of each one of which the reactivity of the chemical system was evaluated by recording the pressure drop within the reactor and by analysing, by gas chromatography, the contents of the atmosphere inside the reactor.

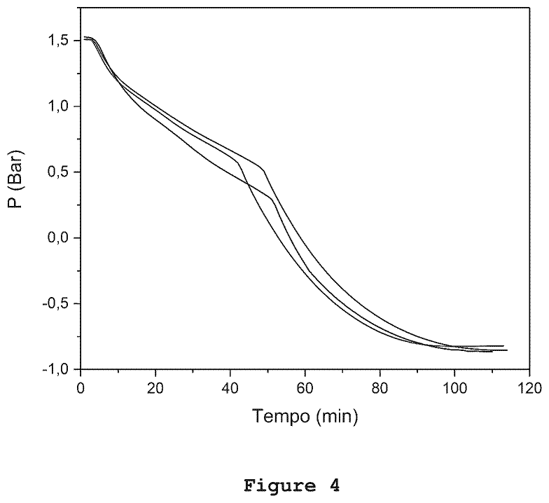

The annexed FIG. 3 shows the trend of CO₂ conversion as a function of milling time.

The data show a sigmoidal profile with an asymptotic value that reaches quantitative conversion (100%) in the tests conducted with 60 to 90 minutes of mechanical treatment.

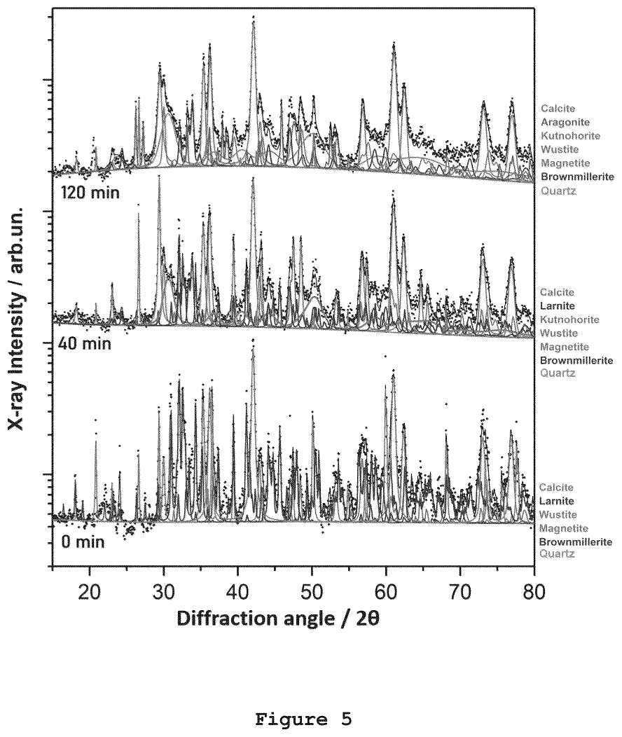

The analysis of the gases inside the milling reactor highlighted that no hydrocarbons were formed, nor formation of molecular hydrogen, H₂, occurred. The annexed FIG. 4 discloses the value of the pressure within the reactor as a function of milling time: the pressure of CO₂ decreases in a monotonous manner, and the slope of the curve changes abruptly after approx. 40 minutes of mechanical treatment under the specific conditions in use, showing an increased gas absorption rate, and the process with an asymptotic trend continues until the system reaches a value of 0.9 bar below the atmospheric pressure; the three data sets shown confirm the reproducibility of the process.

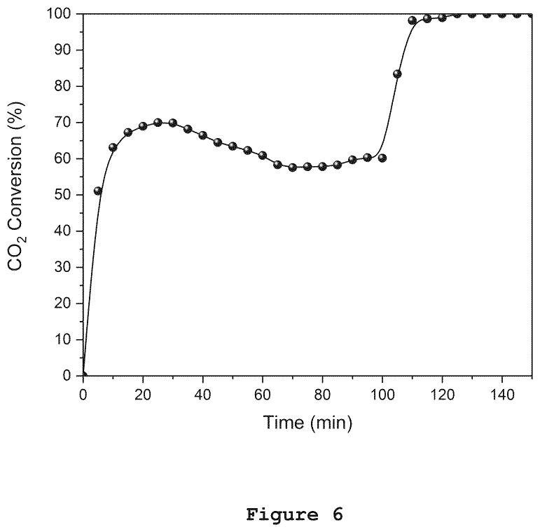

As shown by the diffractogram of the annexed FIG. 5, the starting material is characterized by different crystallographic phases, in particular identified as Quartz (SiO₂), Brownmillerite (Ca₂ (Al, Fe)₂O₅), Larnite (Ca₂SiO₄), Calcite (CaCO₃), Magnetite (Fe₃O₄) and Wustite (FeO).

Notwithstanding the complexity of the diffractogram, the curve of the sample that was treated mechanically for 40 minutes clearly shows the transformation that took place, forming carbonate-type crystallographic phases, and a phase can also be identified which is isomorphic to Kutnohorite (the nominal composition of which is CaMn (CO₃)₂, but which hereinafter will preferably be indicated as Ca,X(CO₃)₂ due to mass balance constraints), while a decrease occurs in Larnite contribution, which can no longer be observed in the curve of the sample treated for 120 minutes.

The relative abundance of the different crystallographic phases in the three samples analysed is summarized in the following Table 1.

TABLE 1
Crystallographic phases observed in preliminary batch test conducted on samples of slags
from EAF processes, and relative abundance thereof as estimated by Rietveld analysis.

| | Larnite | Wustite | Magnetite | Brownmillerite | Quarzo | Calcite | Aragonite | Kutnohorite |
|---|---|---|---|---|---|---|---|---|
| | Ca₂SiO₄ | FeO | Fe₃O₄ | Ca₂(Al, Fe)₂O₅ | SiO₂ | CaCO₃ | CaCO₃ | Ca, X(CO₃)₂ |
| Campione | monoclino | cubico | cubico | ortorombico | trigonale | trigonale | ortorombico | Trigonale |
| 0 min | 49% | 22% | 14% | 3% | 6% | 6% | — | — |
| 40 min | 15% | 22% | 14% | 6% | 2% | 26% | — | 15% |
| 120 min | — | 22% | 14% | 6% | 2% | 20% | 10% | 26% |

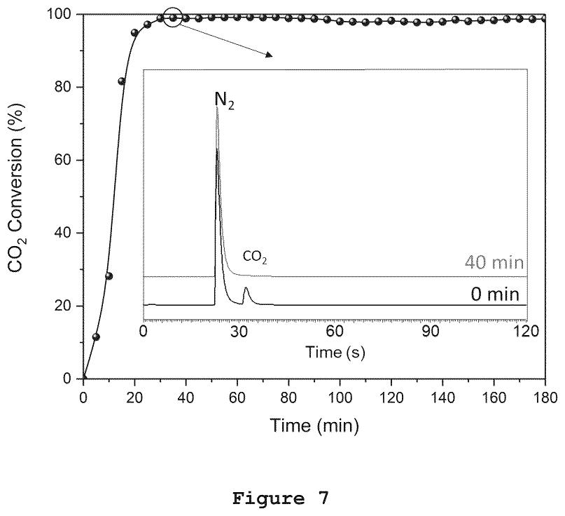

The data obtained indicate that the mechanical treatment, under the present conditions, promotes the reactivity of the system by fostering the conversion of CO₂ as the silicate phase (Larnite) is progressively transformed in the carbonate phases (Calcite, Aragonite and a phase isomorphic to Kutnohorite). Considering such evidence, and in view of possible applications, the next tests were conducted by feeding the mechanochemical reactor in “continuous” mode, i.e., with a mixture of N₂ and CO₂ in a relative N₂:CO₂ ratio of 90:10 under conditions of 5 ml/min flow and atmospheric pressure. The kinetic curve drawn in the annexed FIG. 6 shows high conversion rate during the first phase of mechanical treatment, reaching approx. 70% conversion after approx. 30 minutes of milling, followed by a decrease in the speed and even a reduction in the conversion value.

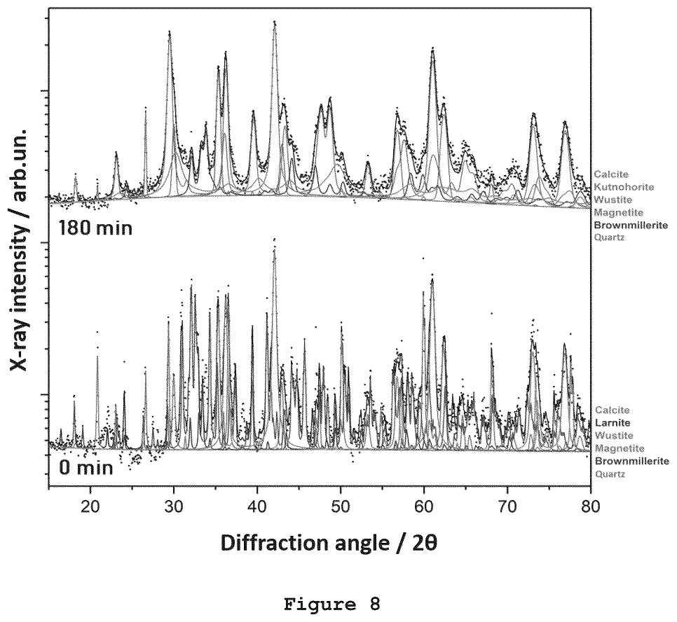

90 minutes after the beginning of the treatment, the slope of the curve increases again quickly, and CO₂ conversion reaches the 100% value and then remains constant until the end of the measurement. The curve seems to indicate a two-stage process, wherein quantitative conversion values are reached after a first phase of activation of the system. At the end of the first stage, the solid phase seems to show a partial evolution of the CaSiO₄ phase towards the formation of a carbonate phase. From an application viewpoint, the confirmation of the possibility of reaching extremely high values of CO₂ conversion in continuous processes led to the execution of more in-depth tests, subjecting the solid and liquid reactants (EAF powders and H₂O) to a mechanical pre-treatment in CO₂ atmosphere for 40 minutes. At the end of the pre-treatment, the mechanochemical reactor was fed with a mixture of N₂ and CO₂ in a relative N₂:CO₂ ratio of 90:10 under conditions of 5 ml/min flow and atmospheric pressure. The test results, expressed as percent values of CO₂ conversion over time, highlighted very good reproducibility characteristics, with a variability percentage of less than 5% (attributable to the experimental/instrumental error), and the annexed FIG. 7 shows a typical profile of the experimental data of the reaction kinetics. The pattern shows a sigmoidal CO₂ conversion profile.

In all tests carried out, conversion grows significantly without showing any appreciable initial induction time. Conversion speed increases, reaches a maximum value at an inflection point in the curve, and then decreases: the asymptotic value towards which the conversion tends exceeds the value of 95%, appearing to be quantitative and remaining almost constant for 3 hours. The sigmoidal pattern of the kinetic curve is typical of many mechanochemical processes, and particularly of processes involving solid-gas heterogeneous systems. Such a pattern has often been interpreted as the result of nucleation-and-growth processes and analysed on the basis of Avrami-Erofeev mechanisms.^{[11]} Without going into details of mechanistic analysis, it is however useful to underline, in this context, that the shape of the kinetic curve profile comprises multiple elementary stages of the conversion process, including superficial H₂O adsorption by the solid phase, dissociation of the same, and processes of H₂ and O₂ scattering in the bulk of the solid phase, adsorption and absorption of CO₂, mineral carbonation processes, processes of scattering and desorption of any gaseous phases formed, and so forth. The exact correlation between such processes and the shape of the curve goes beyond the intention of the present invention, but they should be taken into consideration for a global evaluation of the process.

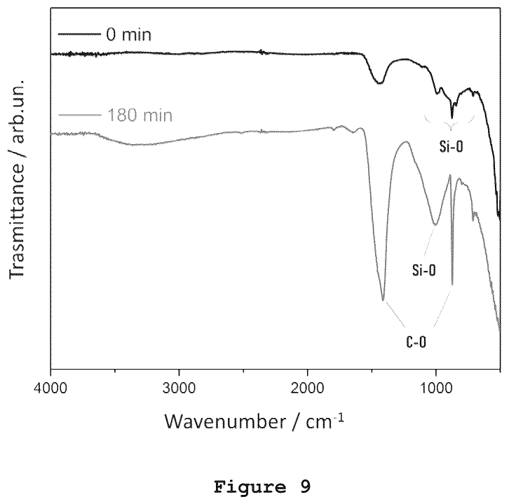

The CO₂ transformation process did not lead, within the test time interval, to formation of any hydrocarbon phases, as had also been observed during the preliminary tests, and in contrast with the results of processes involving other solid phases of the silicate type. Nor was observed in the gaseous mixture contained in the jar, throughout the mechanical treatment, the presence of any molecular H₂. The data obtained from the structural analysis, conducted by X-ray diffractometry and IR spectroscopy, followed the same direction. The annexed FIG. 8 shows the XRD curves relating to the untreated EAF sample and after 180 minutes of mechanical treatment under the previously specified conditions.

The initial sample is characterized by Quartz (SiO₂), Brownmillerite (Ca₂ (Al,Fe)₂O₅), Larnite (Ca₂SiO₄), Calcite (CaCO₃), Magnetite (Fe₃O₄) and Wustite (FeO), and the relative abundance (quantity) values of the different phases are shown in the following Table 2.

TABLE 2
Crystallographic phases observed in tests conducted in flow/continuous mode on samples of
slags from EAF processes, and relative abundance thereof as estimated by Rietveld analysis.

| | Larnite | Wustite | Magnetite | Brownmillerite | Quarzo | Calcite | Kutnohorite |
|---|---|---|---|---|---|---|---|
| | Ca₂SiO₄ | FeO | Fe₃O₄ | Ca₂(Al, Fe)₂O₅ | SiO₂ | CaCO₃ | Ca, X(CO₃)₂ |
| Campione | monoclino | cubico | cubico | ortorombico | trigonale | trigonale | trigonale |
| 0 min | 49% | 22% | 14% | 3% | 6% | 6% | — |
| 180 min | — | 22% | 14% | 6% | 2% | 41% | 15% |

The data pertaining to the sample withdrawn at the end of the mechanical treatment indicate that the relative abundance of the Fe₃O₄ and FeO phases remained almost unchanged (except for the instrumental error), thus confirming that no oxidation process occurred in Fe²⁺ to give Fe³⁺. Likewise, no oxidation processes were observed in other metal ions within the solid phase.

The reactivity of CO₂ under the studied conditions resulted in the formation of carbonates from the Larnite phase, which evolved towards the formation of Calcite and Aragonite phases as well as a phase isomorphic to Kutnohorite, as had been previously observed in the above-mentioned preliminary batch tests.

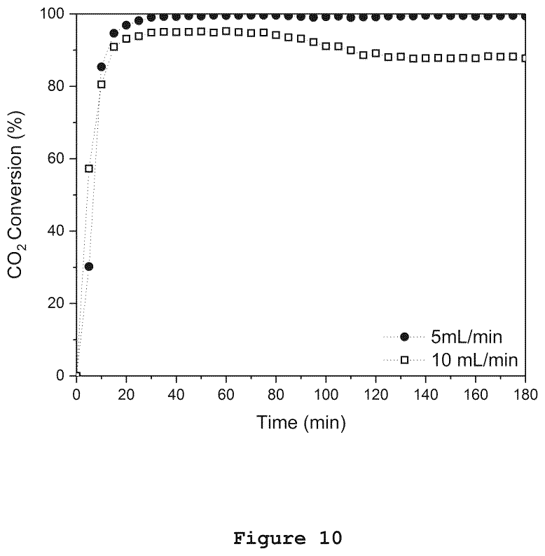

The FT-IR analyses conducted on the solid compounds confirmed the formation of carbonate-type phases, as shown in the annexed FIG. 9.

While the pattern concerning the untreated (initial) sample is characterized by signals that are typical of the SiO₄²⁻ bond and attributable to the CaSiO₄ phase, although there is already a signal that can be attributed to the CO₃²⁻ functional group, the curve pertaining to the sample withdrawn at the end of the mechanical treatment is dominated by signals attributable to the CO₃²⁻ functional group, the relative intensity of which grows considerably. The signal attributable to SiO₂ can also be observed.

In order to deepen the knowledge of the reaction mechanism that governs the process, and to evaluate the experimental conditions with a view to obtaining the best conversion and yield data, the process was studied as a function of a number of parameters, in particular a) the gaseous mixture flow rate value, b) the mechanical energy transferred to the reactants during the mechanical treatment, c) the effect of the pre-treatment.

### a) Dependency of the Kinetics on the Flow Rate Value of the Gaseous Mixture Inside the Mechanochemical Reactor

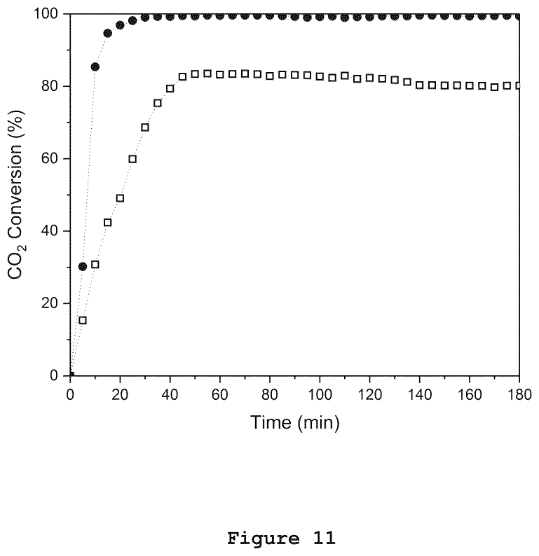

The process of mechanical activation of a chemical system containing solid and gaseous phases is extremely complex, and its kinetic interpretation requires evaluating a large number of parameters, which often cannot be directly acquired in the course of the reaction. The energy transfer resulting from the mechanical action occurs in a discontinuous and punctual manner, producing breaking and comminuting effects on the solid reactants which determine a constant renewal of the surfaces exposed to the reactive atmosphere, resulting in the generation of non-equilibrium states and increased reactivity. Processes of superficial adsorption and absorption, scattering, mass transfer and precipitation of new solid phases are just some of the phenomena that occur in succession during the mechanochemical process, thus making it extremely complex. In this frame, the average value of the time spent inside the mechanochemical reactor is very important, which depends on the rate of supply of the gaseous mixture. As previously mentioned, the tests described herein were carried out using a flow rate of 5 ml/min under the adopted experimental conditions. Any variations of this parameter, up to a value of 10 ml/min, do not imply significant variations in the kinetic trend of CO₂ conversion, expressed as a volume percentage, as can be inferred by comparing the two kinetic curves shown in the annexed FIG. 10, while the total number of CO₂ moles converted during the treatment is almost doubled (from approx. 3.4 mmoles to approx. 6.5 mmoles), and the yield value, expressed as mmoles of converted CO₂ per gram of solid slag, increases from 1.68 to 3.24.

### b) Effect of the Parameters Related to the Mechanical Treatment Dynamics on the Reaction Kinetics

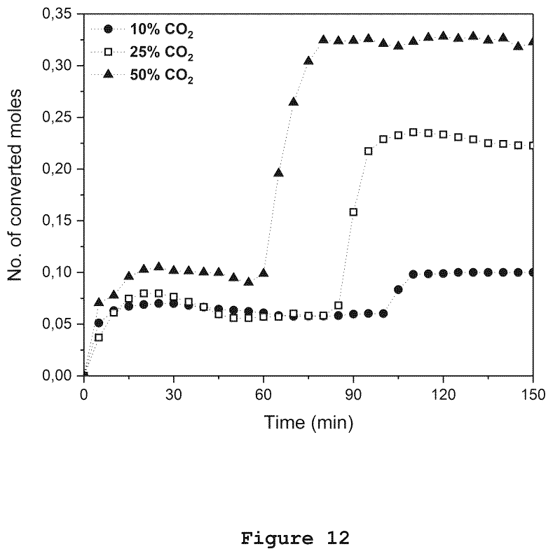

In mechanochemical processes, energy transfer occurs discontinuously during the collisions involving milling bodies and reacting systems: collision frequency, kinetic energy of the milling bodies as the collision is transferred, and time duration of the collisions are key parameters for evaluating the energy and the power transferred during the mechanical action. A variation in the revolution speed of the motor that sets the milling bodies in motion will also determine a change in the transferred kinetic energy (by affecting the relative velocity of the milling bodies) and in the collision frequency; a variation in the mass of the milling bodies will mainly affect the bodies' kinetic energy at the instant of collision. Without describing in detail the absolute evaluation of the energy and intensity transferred during the mechanical treatment, since this goes beyond the scope of the present invention, it may however be of interest to analyse the effects of variations of such parameters on the conversion kinetics. In this regard, the annexed FIG. 11 shows a comparison between the previously shown curve, which concerns the tests conducted using 3 steel balls of 4 g each as milling bodies, with the electric motor operating at 875 revolutions per minute (RPM), and the one obtained from a test conducted with 5 steel balls of 4 g each, with the motor operating at a speed of 745 RPM.

Under these latter conditions, the energy transferred during the collisions is certainly lower than in the previous case, and the decreased revolution speed of the motor does not compensate for the potential increase in the collision frequency resulting from the increased number of milling balls. Notwithstanding this, the reaction kinetics show very high CO₂ conversion values, which reach, even though less quickly than in the previous case, values in excess of 80%, thus confirming again the extremely high reactivity of the mechanically activated slags.

### c) Effect of the Composition of the Gaseous Mixture on the CO₂ Conversion Kinetics

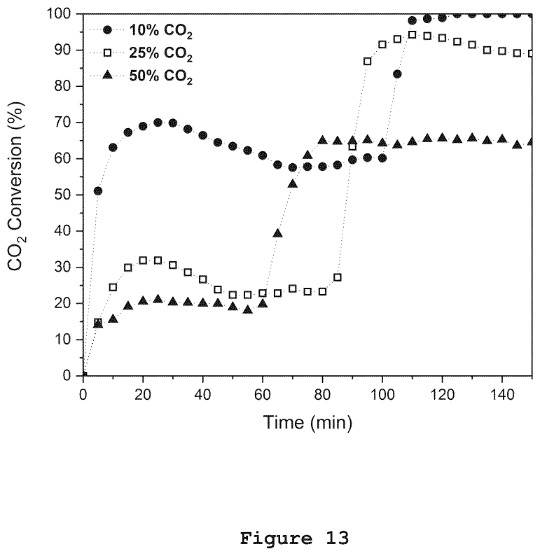

The effect of the chemical composition of the gaseous mixture on the reaction kinetics was evaluated by means of a series of tests conducted without pre-treatment in reactive atmosphere. The kinetic profiles are shown in the annexed FIG. 12, expressed as the number of transformed CO₂ moles as a function of mechanical treatment time, for different compositions.

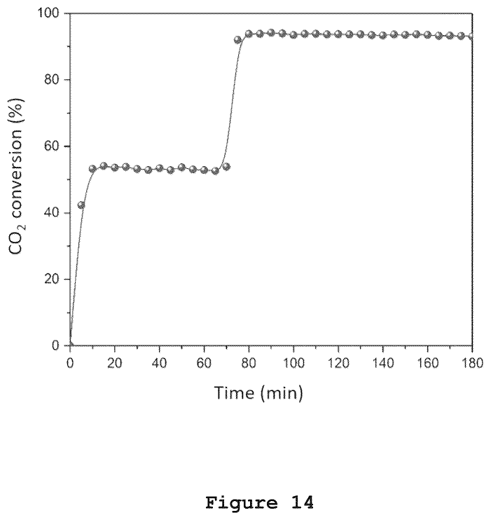

The three curves express the absolute values of the transformed CO₂ moles as a function of time, in the presence of 2 g of EAF slag powders. The observed patterns confirm the existence of a composite reaction mechanism, wherein two stages appear to exist regardless of the composition of the gaseous mixture, which however seems to play the important role of defining the amount of CO₂ converted in the various tests. It must be underlined that the expression of the results of the previous analyses on a percentage scale, shown in the annexed FIG. 13, confirms the extremely high degree of CO₂ conversion independent of mixture composition, highlighting the fact that the one with the lowest CO₂ content provided a 100% conversion, and high values, i.e., approx. 90% and 70%, were obtained from the mixtures containing the highest percentages of CO₂, as indicated in the Figure.

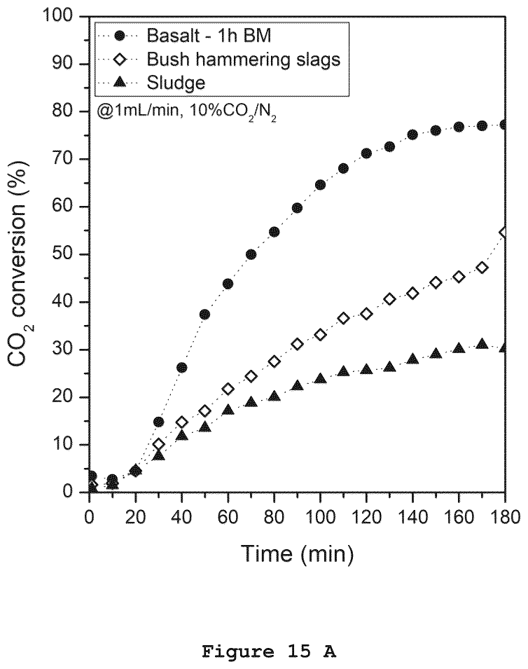

The analysis of the kinetic data about the processes using EAF slags ends with the conversion profile shown in the annexed FIG. 14, which concerns the results observed in tests conducted in the absence of steel milling bodies, wherein 3 EAF residues having a mass of approx. 1 g each were subjected to the mechanical treatment, and the other experimental parameters were kept at the levels of the last tests previously mentioned herein, i.e., N₂:CO₂ mixture=90:10, 5 ml/min, 875 RPM, no pre-treatment.

In this case as well, a pattern can be observed which reminds of a mechanism with two successive reaction stages; however, it should be noted that the mechanical action exerted by the reactants' masses is sufficient to increase the reactivity of the system and reach the quantitative level (100%) of conversion, which remains stable over time within the measurement range.

2) Results of the CO₂ Conversion Process Using Basalt Scraps from Industrial Processes

The study concerned basalt powders produced during different basalt processing stages, namely: a) powders obtained by mechanical treatment of scraps produced during cutting processes; b) powders obtained from product refinement processes using bush-hammering techniques; c) sludges (i.e., wet powders) obtained during the cutting phases with addition of H₂O. The materials thus obtained were employed in CO₂ conversion tests under the previously mentioned experimental conditions, using a gaseous mixture of N₂:CO₂=90:10 with a flow rate of, unless otherwise specified, 1 ml/min.

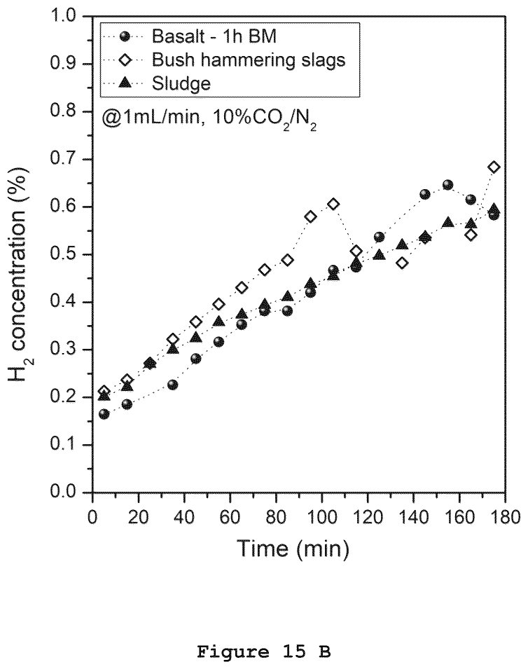

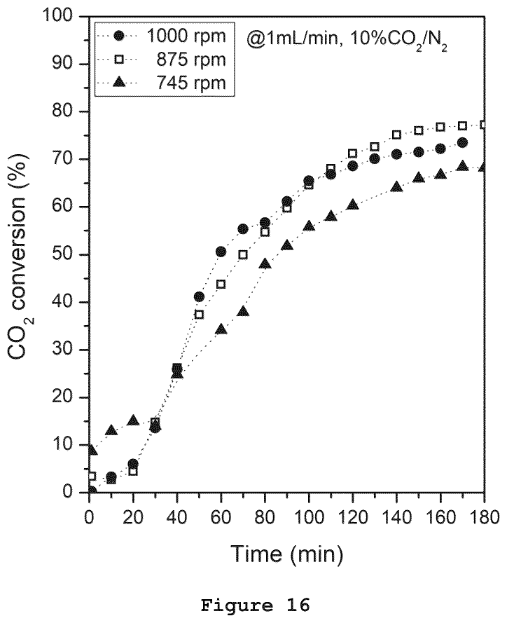

In the annexed FIG. 15A, the kinematic profiles obtained from the tests conducted on these three types of basalt powders show their efficacy in the mechanochemical process of CO₂ conversion, highlighting the different reactivity thereof: in all the three curves, CO₂ conversion values increase over time, showing a sigmoidal pattern, and the highest values were reached in the tests conducted on scrap powders subjected to pre-grinding, which reach, in asymptotic form, a value of approx. 80%. The relationship between the shape of the kinetic curves and the reaction mechanism is probably the same as the one previously observed for the different chemical systems mentioned above, and an in-depth analysis of this result does not fall within the peculiar aspects of the present patent application, while it is worthwhile to point out the effectiveness of the process using basalt scraps from industrial processes, as well as the possible applications thereof.

The CO₂ conversion process using basalt is also accompanied by the evolution of molecular hydrogen, H₂, generated in-situ starting from water (which in this case does not act as a solvent, but as a real reactant), which develops in similar quantities in the tests starting from the three materials of FIG. 15A, and the trend of H₂ concentration inside the reactor during the three tests is shown in the annexed FIG. 15B.

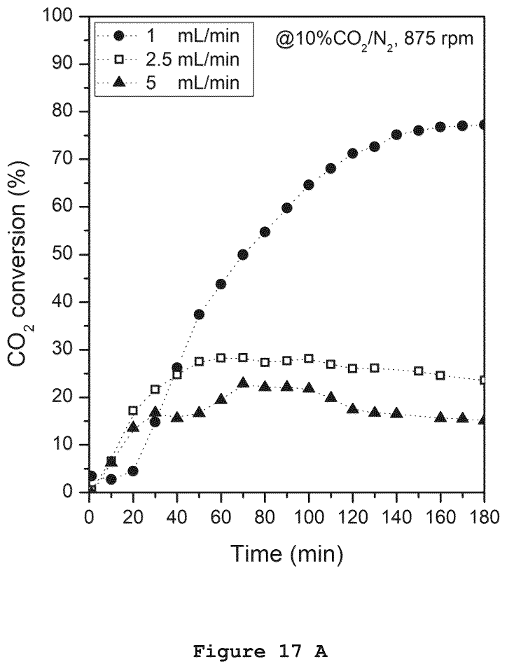

The variation of the dynamic regime of the mechanochemical process, obtained by adjusting the revolution speed of the electric motor of the reactor (the tests were carried out at 1,000, 875 and 745 RPM, respectively) induces no substantial variation in the CO₂ conversion process, as demonstrated by the kinetic data shown in the annexed FIG. 16.

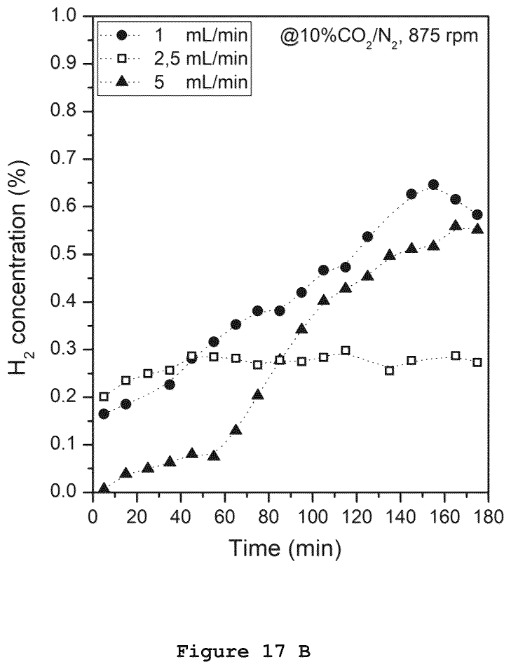

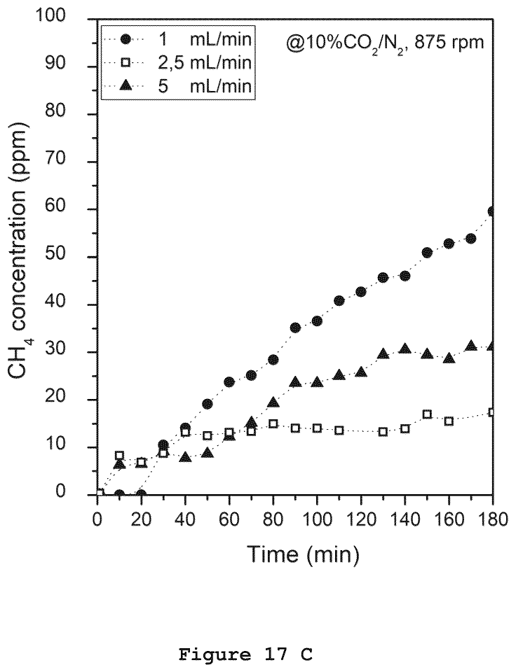

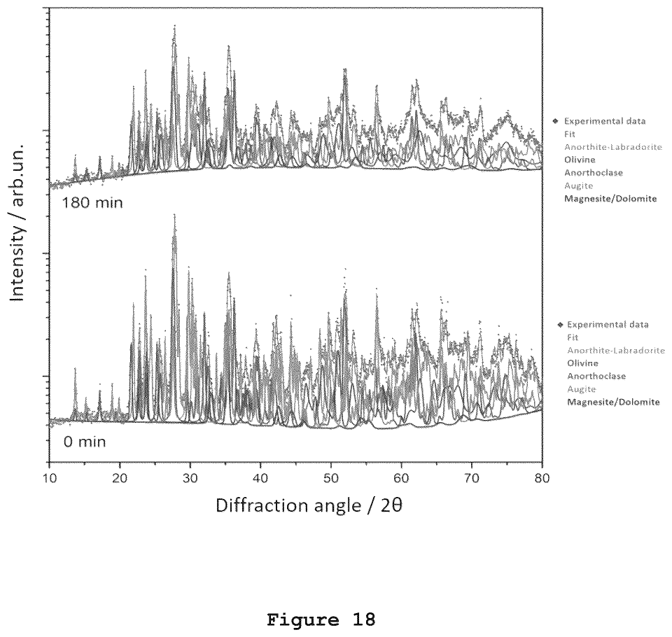

In contrast, a variation in the value of the gaseous mixture flow within the reactor significantly affects the trend of the CO₂ conversion data, which, as shown by the patterns in the annexed FIG. 17A, decreases as the flow rate increases, being respectively 1 ml/min, 2.5 ml/min, 5 ml/min. As previously described herein, the CO₂ conversion process using basalt powders is accompanied by formation of H₂ and also light hydrocarbons, mainly methane,^{[11]} as a consequence of CO₂ hydrogenation. FIGS. 17B and 17C show the trends of the concentration values, inside the reactor, of such gaseous species. The observed values are not high, but there appears to be a correlation with the flow values of the reacting gaseous mixture. The presence of such products also indicates that the mechanism of the CO₂ conversion reaction using basalt powders is different, at least partly, from the one observed on materials coming from residues of EAF processes. In this case, in fact, the reduction of the hydrogen of H₂O to molecular H₂ implies the presence of an oxidation process involving some metal ion in the solid substrate, which process is not observed, on the contrary, on slags from EAF processes.

The XRD structural analysis of the above-mentioned three types of basalt powders revealed that they were composed, prior to the CO₂ conversion tests, of the following crystallographic phases:
Anorthite-Labradorite [(Ca,Na) (Si,Al)₄O₈] (48% by weight),
Anorthoclase [(Na,K)AlSi₃O₈](22%),
Augite [(Ca,Mg,Fe²⁺,Fe³⁺,Ti,Al)₂(Si,Al)₂O₆](17%),
Olivine (Mg,Fe)₂SiO₄ (11.5%),
Magnesite/Dolomite (Ca,Mg) (CO₃)₂ (1.5%).

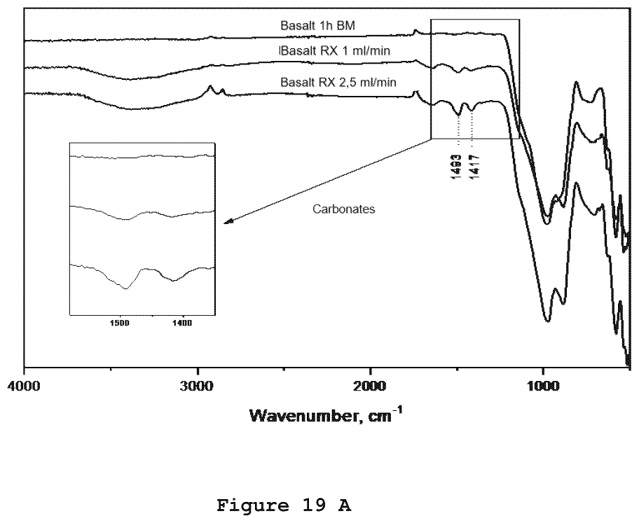

It also turned out that said three types only differ for small relative abundance variations. The curves shown in the annexed FIG. 18 exemplify the diffraction pattern of the samples before and after the mechanical treatment in the presence of CO₂. In particular, the lower diffraction profile in the annexed FIG. 18 refers to basalt powders pre-ground for 1 h, whereas the upper profile refers to powders subjected to the mechanochemical activation process in the presence of CO₂ (180 min, 100% CO₂, 0.3 mL H₂O, 875 RPM).

The diffractograms are per se very complex, and the crystallographic-phase identification and microstructural refinement procedure, conducted using the Rietveld method, made it possible to identify the above-mentioned component phases.

The pattern relating to the sample after the mechanical treatment seems to show some variation in the relative abundance, along with an increased carbonate-based phase, coherently with the chemical process under way; it must however be pointed out that such variation is, in very complex patterns, of the same order of magnitude as the experimental error.

Anorthite-Labradorite [(Ca,Na) (Si,Al)_{4º8]} (39.3% by weight),
Anorthoclase [(Na, K) AlSi₃O₈] (24.6%),
Augite [(Ca,Mg,Fe²⁺,Fe³⁺,Ti,Al)₂(Si, Al)_{2º6]} (17%),
Olivine (Mg,Fe)₂SiO₄ (16.6%),
Magnesite/Dolomite (Ca,Mg) (CO₃)₂ (2.5%).

A comparison between FT-IR analyses conducted on the untreated (initial) powders and on the solid substrate at the end of the CO₂ conversion tests highlights the presence of silicate phases and the formation of carbonate phases, which the XRD analyses only permitted to catch a glimpse of.

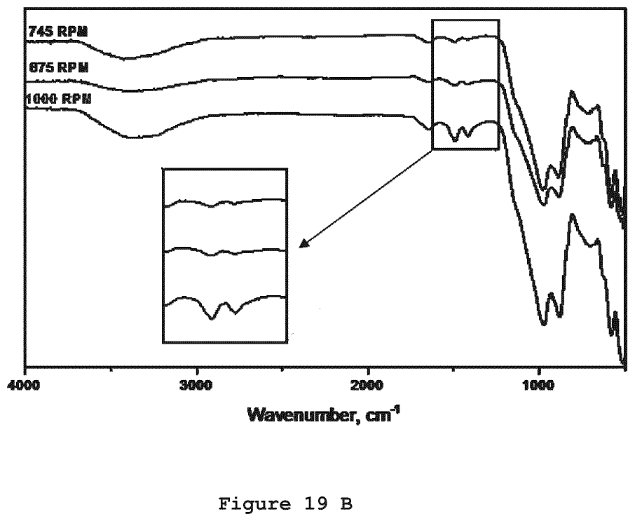

Indeed, the patterns shown in the annexed FIGS. 19A and 19B show signals between 1,000 and 500 cm⁻¹ which can be attributed to stretching and bending vibrations of the Si—O—Si bonds that characterize silicates, which can be easily identified in all spectra, while the samples subjected to mechanical treatment under reactive conditions show signals that are typical of the vibrations of the CO₃²⁻ group in the spectrum region between 1,420 and 1,500 cm⁻¹.^{[12][13]} The intensity of such signals increases with the flow of reactant gas, with the revolution speed of the motor (i.e., with the collision frequency and the kinetic energy transferred to the reactants), and also with the CO₂ fraction in the reactive gaseous mixture (not shown).

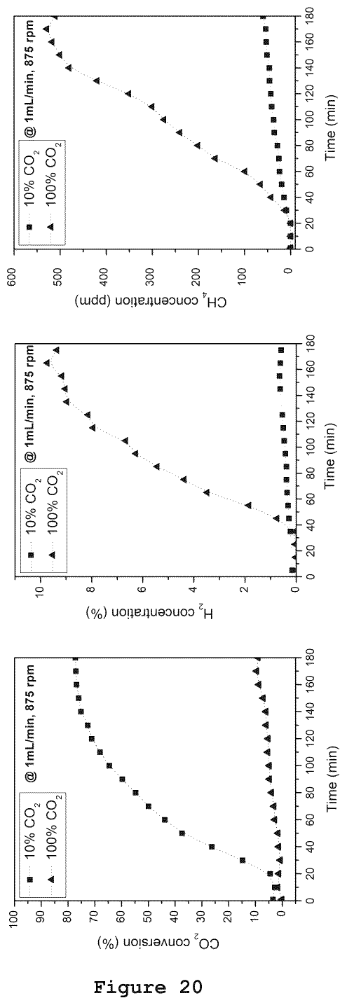

The tests conducted with a different CO₂ content in the gas mixture permit clarifying this aspect. The annexed FIG. 20 shows a comparison between the kinetic patterns recorded with the two compositions of the reactive gaseous phase: the data expressed in percentage terms seem to indicate higher conversion values for the mixture diluted in N₂, but the conversion value expressed in absolute terms, i.e., the number of CO₂ moles converted per time unit, is very similar; and hydrogen concentration, and hence methane concentration, are much higher in the tests carried out with pure CO₂. This result can be interpreted as a confirmation of the mechanistic hypothesis according to which the first stage of the process is CO₂ dissolution into H₂O to form H₂CO₃ and, in the basic environment determined by the existing solid phases, HCO₃⁻. In the presence of oxidation-capable metal ions acting as reducers, the H+ ions of carbonic acid or bicarbonate are reduced to H₂, thereby promoting the precipitation of carbonate salts, while at the same time promoting processes of hydrogenation of unprecipitated CO₂ as metal carbonates. In this context, the higher CO₂ concentration can lead to increased H₂ in the atmosphere of the mechanochemical reactor, with higher concentrations in the form of metal carbonates.

3) Further Results Concerning the CO₂ Conversion Process Using Slags Derived/Obtained from EAF Metallurgical Processes.

As previously reported herein, the study of the reactivity of EAF slags obtained from industrial steelmaking processes highlighted that CO₂ conversion occurs, under the adopted experimental conditions of a chemical process induced by mechanical activation with continuous supply of gas at atmospheric pressure, with relatively fast kinetics, and the analysis of the gases eluted from the mechanochemical reactor shows no formation of either hydrocarbons or molecular hydrogen H₂. The main chemical process of CO₂ conversion can be identified in the formation of solid carbonate phases with formula CaCO₃ (Calcite, Aragonite and a phase isomorphic to Kutnohorite) from the Ca₂SiO₄ silicate phase (Larnite) contained in the starting material.

In order to explore the reactivity of different chemical systems other than raw EAF slags, which may be able to activate CO₂ reduction processes to give high added value systems, the experimental investigation was directed to evaluating composite materials, obtained by adding a metal element, e.g., in a percentage of 10% by weight, to the EAF-type material. The preparation of such materials is justified by the possible different reactivity of the composite systems in comparison with the EAF materials in the CO₂ conversion process, so that the reaction mechanism can be modulated to promote the formation of molecular hydrogen and/or hydrocarbons.

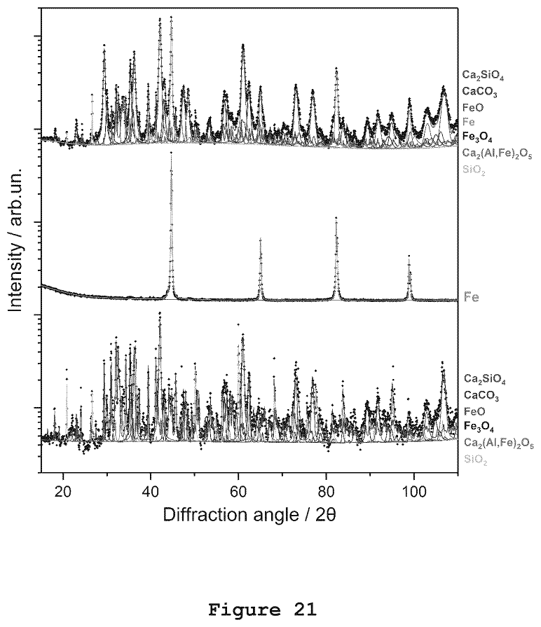

To this end, by way of non-limiting example, three types of composite materials were prepared, respectively characterized by the addition of Fe, Ca and Mg, in elementary form and in a percentage of 10% by weight, to the EAF matrix. The mixed powders were then subjected to the mechanochemical CO₂ conversion tests. The annexed FIGS. 21, 22 and 23 show, respectively, the data of the X-ray diffraction analyses conducted on the three systems mentioned above. In particular, FIG. 21 refers to the EAF/Fe(10%) composite system, wherein the pattern at the bottom is the diffractogram of the EAF material already described in FIG. 5. The middle pattern is the X-ray diffraction pattern of raw metallic Fe, while the pattern at the top is the diffractogram of the composite material at the end of the mechanochemical CO₂ transformation test.

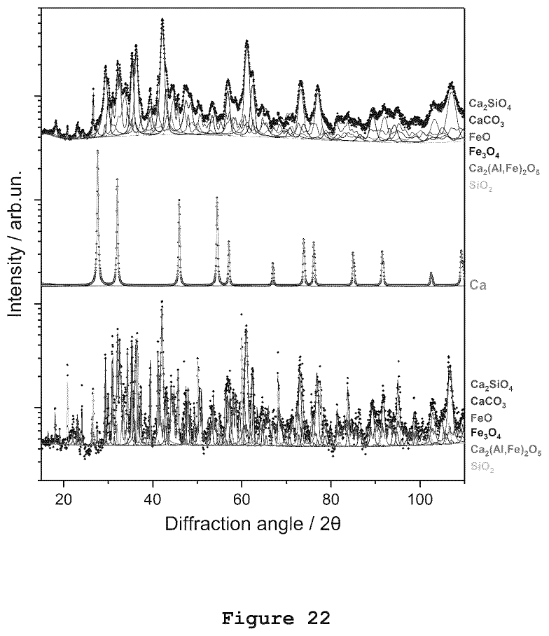

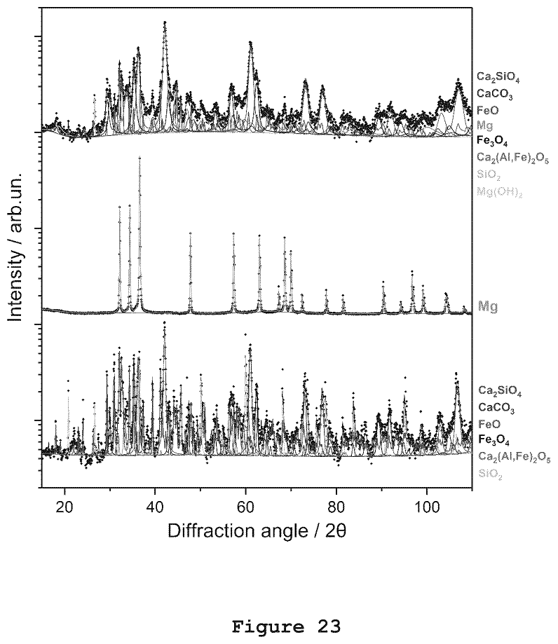

In turn, FIGS. 22 and 23 show, respectively, the corresponding X-ray diffraction (XRD) analyses of the other two systems under examination, i.e., the EAF/Ca(10%) and EAF/Mg(10%) composite materials.

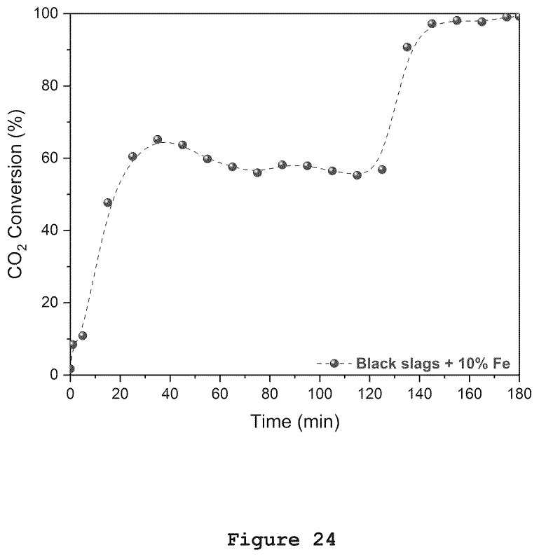

As far as the EAF/Fe (10%) system is concerned, the CO₂ transformation process, conducted under the same experimental conditions as those adopted in the tests conducted on the raw EAF material (i.e., a gaseous mixture of CO₂:N₂=10:90 V/V, gas supplied to the reactor at 5 ml/min, mill revolution speed of 875 RPM), was studied as a function of time, and the corresponding kinetic curve is shown in the annexed FIG. 24. The profile shown in said Figure appears to be superimposable on the kinetic curves of the same process conducted in the presence of the raw EAF material: the pattern suggests a 2-stage mechanism, as was already observed before, wherein the second stage begins after approx. 100 minutes of mechanical treatment. The final asymptotic pattern testifies the quantitative CO₂ conversion. Moreover, CO₂ conversion is not accompanied by either production of molecular H₂ or formation of hydrocarbons. The presence of Fe does not seem to affect the reaction mechanism, and this is also testified by the XRD analysis of the solid material at the end of the process, where the same crystallographic phases as those obtained at the end of the test on raw EAF can be observed, in this case accompanied by the crystallographic peaks that are typical of elementary Fe.

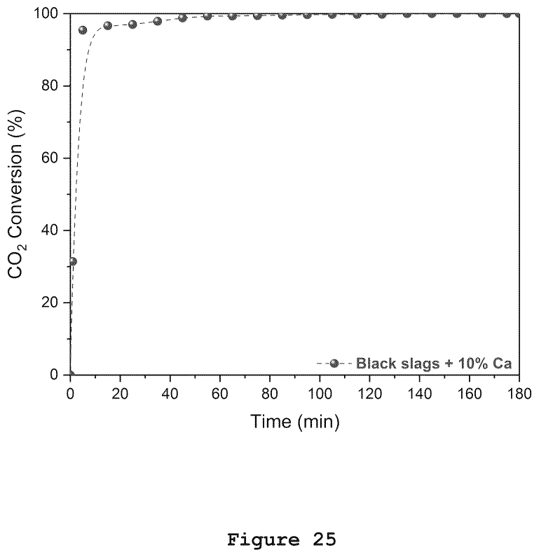

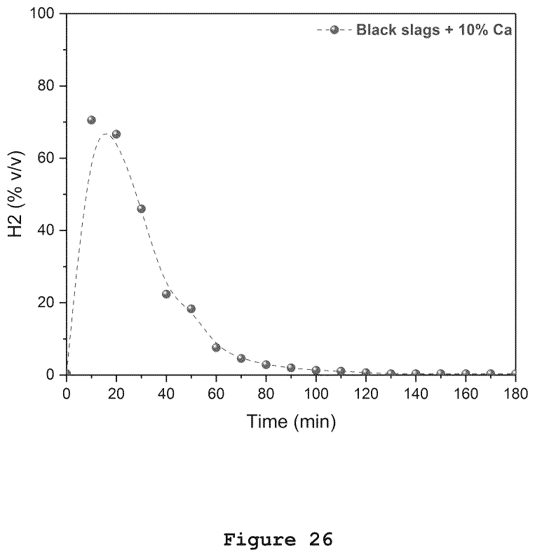

In its turn, the sample of EAF/Ca(10%) composite shows a different kinetic profile, as shown in the annexed FIG. 25. In this case, CO₂ conversion does not show a profile that is typical of a two-stage mechanism; on the contrary, it is characterized by very fast kinetics reaching an asymptotic pattern, corresponding to a quantitative value of CO₂ conversion, as early as after just a few minutes of treatment under the adopted experimental conditions, and remains unchanged throughout the test. CO₂ conversion is accompanied by H₂ evolution, with formation kinetics as shown in the annexed FIG. 26. In this case, H₂ formation is observed already at the very first instants of mechanical treatment, with a rapid increase that reaches a maximum concentration in the gaseous mixture exiting the reactor after approx. 15-20 minutes. The concentration of H₂ then decreases to zero after approx. 100 minutes of treatment. The production of H₂, which necessarily occurs via an H₂O reduction process, can be correlated with the available amount of a reducing agent, i.e., Ca, in the composite material. This demonstrates the active role played by metallic Ca in the composite material, which defines and limits the quantity of H₂ that can be obtained during the process. The diffraction data confirm the oxidation of Ca, which is completely transformed into CaCO₃. Lastly, it should be noted that this composite did not lead, under the adopted conditions, to the formation of hydrocarbons following CO₂ reduction.

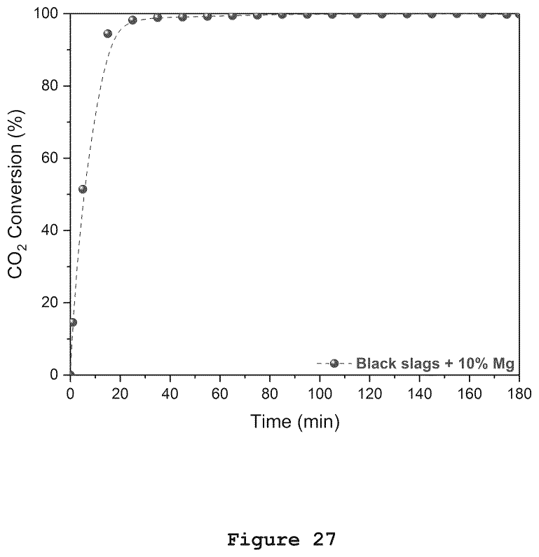

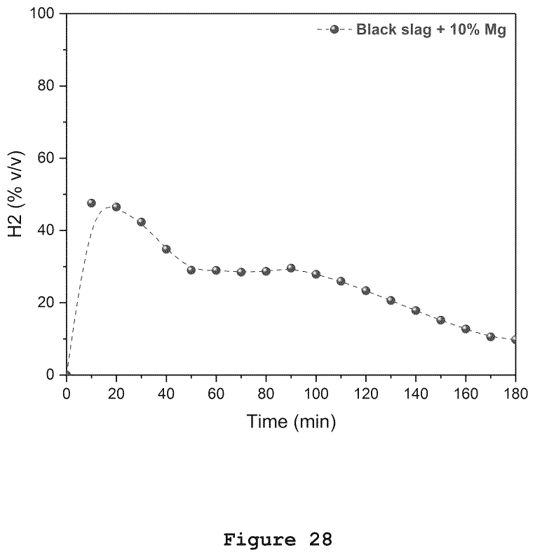

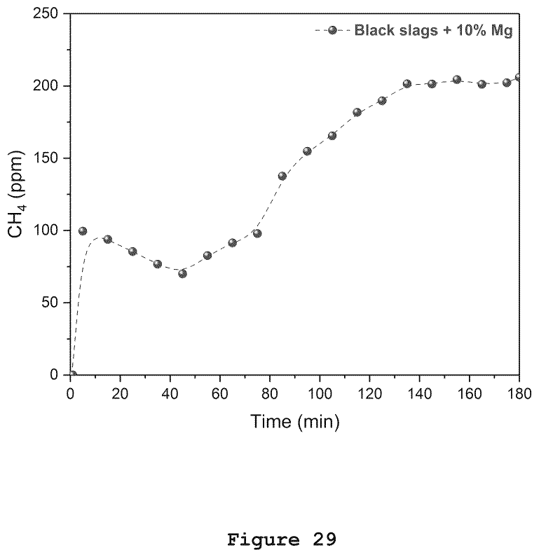

The third system under examination, EAF/Mg(10%), showed CO₂ conversion kinetics comparable with those observed for the preceding EAF/Ca(10%) system. The corresponding CO₂ conversion kinetic profile is shown in the annexed FIG. 27. In this case as well, CO₂ conversion is accompanied by H₂ formation, the pattern of which as a function of time is shown in the annexed FIG. 28. The formation of H₂ in the gas mixture exiting the reactor occurs already at the very first instants of the milling process, and grows rapidly until it reaches a maximum value (approx. 50% V/V) after approx. 15 minutes of treatment. The concentration then decreases, although less quickly than what was observed for the previous system. Unlike the latter, the EAF/Mg(10%) system promotes the production of light hydrocarbons, as shown in the annexed FIG. 29, which illustrates the CH₄ formation concentration profile as a function of time, as measured in the gaseous mixture eluted from the reactor. The profile analysis indicates that CH₄ formation starts already at the very beginning of the milling process and grows in a way similar to the trend of H₂ concentration. CH₄ concentration reaches approx. 100 ppm after approx. 10 minutes of treatment, followed by a slight decrease and then another increase starting from approx. 80 minutes of treatment. The growth continues with a logarithmic trend throughout the observation interval, up to approx. 180 minutes of treatment. The observed CH₄ concentration values are certainly of interest and prove the existence of a CO₂ reduction process giving substances which can be reused as fuels. However, such concentration values do not justify, in absolute terms, the CO₂ conversion data and the H₂ concentration values previously mentioned herein; in fact, the main fraction of CO₂ is converted, in this case as well, as Ca carbonate, as indicated in the upper diffractogram of FIG. 23, which also shows the Mg(OH)₂ phase, which is to be attributed to the redox activity of Mg in the EAF/Mg(10%) composite material.

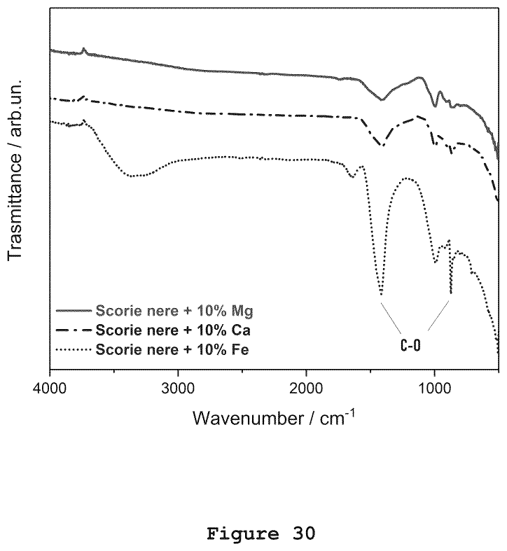

The presence of the carbonate phases in the three solid systems at the end of the mechanochemical tests was confirmed by FT-IR analyses conducted on the respective powders, as shown in the annexed FIG. 30. Signals attributable to vibrational motions of the CO functional group, which are indicative of the presence of carbonate-type phases, are highlighted in FIG. 30, and can be found in the spectra of all the three systems. In particular, said signals attributable to the CO group appear to be more intense for the EAF/Fe(10%) system, wherein there is also a wide signal in the interval from 3,700 to 3,000 cm⁻¹, which can be attributed to the vibrational motions of the hydroxylic group contained in H₂O, which appears to not have been involved in chemical transformation during the treatment, unlike what happened when the treatment was carried out in the presence of the EAF/Mg(10%) and EAF/Ca(10%) systems.

In conclusion, the data obtained refer to a study of the reactivity, induced by mechanical treatment, of composite systems obtained by addition of metals to the raw EAF materials as regards CO₂ transformation. The results of the experimental activity indicate that EAF/ME composite systems (where, by way of example and without limitation, ME=Fe, Ca, Mg, in relative quantities EAF/ME=90/10(%) W/W) show chemical reactivity towards CO₂ under mechanical treatment conditions, each one of them having specific characteristics that are different from the EAF system as such. The EAF/Ca and EAF/Mg systems appear to be active, unlike the raw EAF system and the EAF/Fe system, in promoting redox processes leading to the production of H₂ and, respectively, H₂ and hydrocarbons. Furthermore, they are characterized by faster conversion kinetics approaching quantitative CO₂ conversion in an asymptotic manner.

### CONCLUSIONS

In view of all the above considerations, the core of the present invention can be summarized through the following points, which are listed below for clarity's sake and by way of example, without them being by any means limiting for those skilled in the art.

[1] A continuous mechanochemical process under gas flow (and in the absence of any non-aqueous solvents) to carry out CO₂ conversion into a mixture of high added value chemical compounds, said mixture substantially comprising a mixture of low molecular weight hydrocarbons, such as, preferably, methane, ethylene and ethane, and/or solid products of mineral carbonation, such as, preferably, Ca, Mg and Fe carbonates, wherein said process comprises at least the following phases:
a)—continuously passing a gas flow comprising, or consisting of, CO₂ through a reactor having milling means, and in the presence of reactants consisting of powders of industrial processing slags (wherein said powders contain different/various phases of Ca, Mg and Fe silicates), and possibly of water (whether deionized or not and in liquid or gaseous form), at a temperature ranging from room temperature to ≤100° C.; said reactor is subjected to motion in such a way that the milling means trigger a mechanochemical reaction that, on one side, produces hydrogen from the water, and, successively, said hydrogen thus generated carries out the conversion of one portion of CO₂ into said low molecular weight hydrocarbons, while, on the other side, the other portion of CO₂, or CO₂, reacts with the transformation products of said industrial processing slags and with the non-reacted H₂O to give said solid products of mineral carbonation;
b)—separating and recovering from the gas mixture exiting the reactor said low molecular weight hydrocarbons and non-reacted hydrogen obtained in step a);
c)—separating and recovering from the reactor, at the end of the reaction, said solid products of mineral carbonation obtained in step a).

[2] A process in accordance with the preceding point [1], wherein said reactor is a mill, or a jar, having milling means consisting of rotating spherical bodies (spheres); said mill and said spheres are, preferably, made of an abrasion resistant material, e.g. hardened stainless steel.

[3] The process according to the preceding points [1] or [2], wherein the movement which the reactor is subjected to is a rotary movement at a speed ranging from 500 rpm to 1,500 rpm.

[4] The process according to the preceding point [3], wherein said rotary movement is selected with a speed ranging from 600 rpm to 1.400 rpm.

[5] The process according to anyone of the preceding points from [1] to [4], wherein the gas flow comprising CO₂ contains amounts thereof similar to the ones in the exhausted fumes coming out from post-combustion chimneys of industrial processes; said amounts of CO₂ range from approx. 4% to 20% by volume; more preferably, from 5% to 18%; more preferably, from 6% to 15%; even more preferably, from 7% to 13%; in a particularly preferred embodiment, from 8% to 10% by volume.

[6] The process according to anyone of the preceding points from [1] to [5], wherein said industrial processing slags comprise processing slags from steelmaking processes, e.g., EAF (Electric Arc Furnace), BOF (Basic Oxygen Steelmaking) processes as previously described herein, and other similar processes well known and commonly used in the art, and/or basalt processing scraps.

[7] The process according to anyone of the preceding points from [1] to [6], wherein the highest measured concentrations of the low molecular weight hydrocarbons produced in step a) are about 680 ppm for methane, about 280 ppm for ethane, and about 100 ppm for ethylene, wherein concentrations are expressed in ppm V/V in the gas mixture exiting the reactor.

[8] The process according to the preceding point [7], wherein, in the production of low molecular weight hydrocarbons, methane is the main product with a selectivity of 60%, while the selectivity values for ethane and ethylene are 20-25% and 8-10%, respectively.

[9] The process according to the preceding points [7] or [8], wherein the global yield in methane production is 50%, 10-15% for ethane, and about 10% for ethylene.

[10] Use of the process according to anyone of the preceding points from [1] to [9] to carry out CO₂ conversion into a mixture of high added value chemical compounds, said mixture comprising a mixture of low molecular weight hydrocarbons, mainly methane, ethylene and ethane, and/or solid products of mineral carbonation, mainly Ca, Mg and Fe carbonates, obtained from the silicates of said metals.

### INDUSTRIAL APPLICABILITY OF THE INVENTION

The method of the present invention has made it possible to convert carbon dioxide (CO₂) into high added value chemical compounds under continuous gas flow conditions. In particular, said method converts CO₂ into a mixture of high added value chemical compounds comprising, preferably, low molecular weight hydrocarbons, such as methane, ethylene and ethane, and/or products of mineral carbonation, such as Ca, Mg and Fe carbonates. Said CO₂ conversion is achieved through a mechanochemical process which, being executed in continuous mode, is advantageously applicable to all the activity (particularly the industrial ones) emitting into the atmosphere exhaust fumes comprising, among other possible gaseous substances, significant amounts of CO₂.

### LIST OF REFERENCES

^{[1]} https://www.climate.gov/news-features/understanding-climate/climate-change-atmospheric-carbon-dioxide
^{[2]} J. Wanga et al., International Ocean and Polar Engineering Conference, Sapporo, Japan, June 10-15 (2018)
^{[3]} https://climate.nasa.gov/effects/
[4] M. Rosa et al., Journal of CO2 Utilization, 9 (2015) 82-102
^{[5]} A. S. Agarwal et al., ChemSusChem 4 (2011) 1301-1310
^{[6]} Hydrogen in a low-carbon economy, Committee on Climate Change, London, November 2018
^{[7]} A. Álvarez et al., Chem. Phys. Chem., 18 (2017) 3135-3141
^{[8]} C. Oze et al., Geophysical Research Letters—32 (2015) L10203
^{[9]} F. Wang et al., Minerals Engineering—131 (2019) 185-19
^{[10]} M. McCollom et al, Geochem. Cosmochim. Acta 65 (2001) 3769-3778
^{[11]} V. Farina et al., Front. Energy Res., 7 (2019) 107
^{[12]} E. Turianicová et al, «A POSSIBLE WAY TO STORAGE CARBON DIOXIDE ON MECHANICALLY ACTIVATED OLIVINE (Mg,Fe)2SiO4,»INCOME 2008, Jamshedpur, India, 2008
^{[13]} F. Torre et al, Powder Technology, 364 (2020) 915-923

1. A continuous mechanochemical process under gas flow to carry out CO₂ conversion into a mixture of high added value chemical compounds, said mixture comprising a low molecular weight hydrocarbon mixture, methane, ethylene and ethane, and/or solid products of mineral carbonation, Ca, Mg and Fe carbonates, said process comprising at least the following steps:
a)—continuously passing a gas flow comprising, or consisting of, CO₂ through a reactor having milling means, and in the presence of reactants consisting of powders of industrial processing slags, wherein said powders contain different/various phases of Ca, Mg and Fe silicates, and of water, deionized or not and in liquid or gas form, at a temperature from room temperature to ≤100° C.; said reactor being subjected to motion in such a way that the milling means trigger a mechanochemical reaction that, on one side, produces hydrogen from the present water, and, successively, said hydrogen carries out the conversion of one portion of CO₂ into said low molecular weight hydrocarbons, while, on the other side, the remaining portion of CO₂ reacts with the transformation products of said industrial processing slags and with the non-reacted H₂O to give said solid products of mineral carbonation;
b)—separating and recovering from the gas mixture exiting the reactor said low molecular weight hydrocarbons and non-reacted hydrogen obtained in step a);
c)—separating and recovering from the reactor, at the end of the reaction, said solid products of mineral carbonation obtained in step a).

2. The process according to claim 1, wherein said reactor is a mill, or a jar, having milling means consisting of rotating spherical bodies (spheres); said mill and said spheres being made of hardened stainless steel.

3. The process according to claim 1, wherein the movement which the reactor is subjected to is a rotary movement at a speed ranging from 500 rpm to 1,500 rpm.

4. The process according to claim 3, wherein said rotary movement occurs at a speed ranging from 600 rpm to 1,400 rpm.

5. The process according to claim 1, wherein the gas flow comprising CO₂ contains amounts thereof that are similar to the ones in the exhausted fumes coming out from the post-combustion chimneys of industrial processes; said amounts of CO₂ ranging from about 4% to 20% by volume; in a particularly preferred embodiment, from 8% to 10% by volume.

6. The process according to claim 1, wherein said industrial processing slags comprise processing slags from steelmaking processes, EAF (Electric Arc Furnace), BOF (Basic Oxygen Steelmaking), and the ones that are similar and well known in the art, and/or basalt processing scraps.

7. The process according to claim 1, wherein the highest measured concentrations of the low molecular weight hydrocarbons produced in step a) exiting the reactor are about 680 ppm V/V for methane, about 280 ppm V/V for ethane, and about 100 ppm V/V for ethylene.

8. The process according to claim 7, wherein methane is the main product with a selectivity of 60%, while the selectivity values for ethane and ethylene are 20-25% and 8-10%, respectively.

9. The process according to claim 7, wherein the global yield in methane production is 50%, 10-15% for ethane and about 10% for ethylene.

10. A method for carrying out CO₂ conversion into a mixture of high added value chemical compounds using the process according to claim 1; wherein said mixture comprising a low molecular weight hydrocarbon mixture, methane, ethylene and ethane, and solid products of mineral carbonation, Ca, Mg and Fe carbonates.
